(19) United States
(12) Patent Application Publication
(10) Pub. No.: US 2012/0148661 A1
(43) Pub. Date: Jun. 14, 2012

(54) HIGH BIOAVAILABILITY ORAL PICOPLATIN ANTI-CANCER THERAPY

(75) Inventors: Angelica F. Phillips, Lynnwood, WA (US); Robert H. Earhart, JR., Bainbridge Island, WA (US); Michael S. Perry, Palo Alto, CA (US)

(73) Assignee: Poniard Pharmaceuticals, Inc., Seattle, WA (US)

(21) Appl. No.: 13/264,343

(22) PCT Filed: Mar. 11, 2010
(86) PCT No.: PCT/US10/00735

§ 371 Date: Feb. 27, 2012

Related U.S. Application Data
(60) Provisional application No. 61/169,679, filed on Apr. 15, 2009.
(60) Provisional application No. 61/170,487, filed on Apr. 17, 2009.

(51) Int. Cl. A61K 31/555; A61K 9/14; A61K 9/127; A61P 35/00; A61N 5/00; A61K 9/48; C07F 15/00; B82Y 5/00

(52) U.S. Cl. 424/450; 514/188; 424/452; 424/490; 546/12; 600/1; 977/773; 977/915

(57) ABSTRACT

The invention provides a method of treatment of cancer, wherein a individual doses of picoplatin, each of less than about 200 mg picoplatin content, the individual doses having high oral bioavailability, are administered to a patient in need thereof. The oral bioavailability can be greater than about 50%, or greater than about 75%, or greater than about 90%, depending upon the particular dosage form and dosing regimen used. The invention provides a quasi-metronomic dosing schedule including drug dosing intervals and drug intermission intervals, optionally including fasting periods prior to and following administration of each individual dose of picoplatin.

### CROSS-REFERENCE TO RELATED APPLICATIONS

This application claims the priority of U.S. provisional applications Ser. No. 61/169, 679, filed Apr. 15, 2009, and Ser. No. 61/170,487, filed Apr. 17, 2009, which are incorporated herein by reference in their entireties.

### BACKGROUND

Picoplatin is a new-generation organoplatinum drug that has promise for treatment of various types of malignancies, including those that have developed resistance to earlier organoplatinum drugs such as cisplatin and carboplatin. Picoplatin has shown promise in the treatment of various kinds of cancer or tumor, including small cell lung cancer, colorectal cancer, and hormone-refractory prostate cancer.

Structurally, picoplatin is:

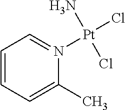

and is named cis-amminedichloro(2-methylpyridine)platinum(II), or alternatively [SP-4-3]-ammine(dichloro)(2-methylpyridine)platinum(II). The compound is a square planar complex of divalent platinum that is tetracoordinate and has three different ligand types. Two ligands are anionic, and two are neutral; therefore as the platinum in picoplatin carries a +2 charge, picoplatin is itself a neutral compound and no counterions need be present. The name “picoplatin” referring to the presence of α-picoline (2-methylpyridine) in the molecule is the United States Adopted Name (USAN), the British Approved Name (BAN), and the International Nonproprietary Name (INN) for this material. Picoplatin is also referred to in the literature as NX473, ZD0473, and AMD473, and is disclosed in U.S. Pat. Nos. 5,665,771, 6,518,428, PCT US2008/001746, filed Feb. 8, 2008, and U.S. Ser. No. 10/276,503, which are incorporated by reference herein.

Tetracoordinate square planar platinum (II) are well known to be subject to oxidation to octahedral Pt(IV) complexes, such as with molecular chlorine. Also, it is well known that square planar platinum (II) complexes are subject to axial attack in ligand displacement reactions by various nucleophiles such as halides, amines, thio compounds, and under some conditions, water. Therefore, while picoplatin is relatively stable in pure form, in the absence of light, it can be subject to degradation under certain conditions, such as in the presence of nucleophilic molecular entities. See Advanced Inorganic Chemistry, F. Albert Cotton and Geoffrey Wilkinson, Second Revised Edition (1966) and later editions, Interscience Publishers. When administered to patients, picoplatin is believed to undergo transformation to some extent to two distinct aqua forms resulting from displacement of either of the chloride ligands. In addition to picoplatin, these cationic species (resulting from displacement of a chloride anion by neutral water) are also able to interact with cellular DNA to bring about cross-linking and eventual cell death. Picoplatin is also known to be unstable in the presence of certain metal oxides, such as iron oxide.

Picoplatin has previously been provided to patients in solution by intravenous (IV) administration. Picoplatin under standard conditions is a solid, and has only sparing solubility in water. The relatively low solubility of picoplatin in water (less than 1 mg/mL) necessitates that substantial volumes of liquid be delivered intravenously to provide a patient with total doses in the range of 100 mg and more (i.e., at a concentration of 0.5 mg/mL, some 200 mL of liquid must be introduced by IV infusion to provide a 100 mg dose). As typical human dosages for cancer patients can be on the order of several hundred milligrams per administration, and may be repeated every few weeks, substantial volumes of liquid must be delivered to the patient for each administration of the substance by the IV route. Intravenous administration is thus undesirable due to the need for needle insertion into a vein, and the relatively prolonged periods over which the patient must be immobile to allow for infusion of the relatively large volumes of the picoplatin solutions.

Picoplatin has been shown to be orally bioavailable in animals to some extent, but its low solubility in water, cytotoxicity and teratogenicity pose obstacles to the preparation of effective oral dosage forms. It has been disclosed that an oral bioavailability of picoplatin of about 10-50% can be achieved. For example, see PCT patent publication number WO2008/097658 by the inventors herein, which is incorporated by reference herein. However, a higher oral bioavailability is desirable.

### SUMMARY

The invention is directed in various embodiments to a method of treatment of cancer using one or more unit dosage forms of picoplatin adapted for oral administration, wherein an unexpectedly high oral bioavailability of the picoplatin is achieved, and to dosage forms adapted for use in a method of the invention. It has been surprisingly discovered by the inventors herein that certain methods of oral administration of picoplatin to cancer patients provides an unexpectedly high bioavailability of the picoplatin in a patient's bloodstream.

It has unexpectedly been found that the oral bioavailability of smaller amounts of picoplatin, less than about 200 mg per individual dose, whether given as one or a plurality of unit dosage forms, is much higher than the oral bioavailability of individual doses containing 200 mg or more of picoplatin. For example, the present regimen provides oral bioavailability of the picoplatin in the patient after ingestion that is greater than about 50%. In various embodiments, the oral bioavailability can be greater than about 75%, or greater than about 90%. For example, as shown below in Table 3, below, oral bioavailability of an individual dose of 50 mg is greater than oral bioavailability of an individual dose of 100 mg, which is greater than oral bioavailability of an individual dose of 200 mg, which is greater than oral bioavailability of an individual dose of 300 mg or 400 mg.

In various embodiments, the invention provides a method of treating cancer in a human patient afflicted therewith, comprising administering orally to the patient one or more individual doses per day each comprising picoplatin, wherein each individual dose comprises less than about 200 mg of picoplatin, wherein an aggregate daily dose comprises a sum of the one or more individual doses administered within a single day, provided that when more than one individual dose makes up an aggregate daily dose, each individual dose is administered non-concurrently with each other individual dose over the course of the day. In various embodiments, an oral bioavailability of each individual dose is greater than about 50%, or a daily average oral bioavailability of the picoplatin to the patient from the daily aggregate dose is greater than about 50%, or both. In various embodiments, the method can further comprise administration of the individual doses throughout a drug dosing interval comprising a successive or intermittent plurality of days. In various embodiments, the method can further comprise quasi-metronomic dosing comprising administering the picoplatin to the patient throughout a plurality of drug administration cycles comprising a duration of treatment, each cycle comprising administering the individual doses, each comprising less than about 200 mg of picoplatin, throughout a drug dosing interval comprising one or more days, followed by a respective drug intermission interval comprising one or more days.

In various embodiments, the invention provides a method of treating cancer in a human patient afflicted therewith, comprising administering orally to the patient one or more individual doses per day each comprising picoplatin, wherein each individual dose comprises less than about 200 mg of picoplatin,

wherein an aggregate daily dose comprises sum of the one or more individual doses administered within a single day, provided that when more than one individual dose makes up an aggregate daily dose, each individual dose is administered non-concurrently with each other individual dose over the course of the day; wherein an oral bioavailability of each individual dose is greater than about 50%, or wherein a daily average oral bioavailability of the picoplatin to the patient from the daily aggregate dose is greater than about 50%;

the method further comprising administration of the individual doses throughout a drug dosing interval comprising a successive or intermittent plurality of days; the method further comprising administering the picoplatin to the patient throughout a plurality of drug administration cycles comprising a duration of treatment, each cycle comprising administering the individual doses throughout a drug dosing interval comprising one or more days, followed by a respective drug intermission interval comprising one or more days;

the method comprising one or more drug dosing intervals wherein in at least one of the drug dosing intervals comprises administration of a boost dose on a first day and of a maintenance dose on one or more following days of each dosing interval;

wherein each individual dose comprises a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant;

or comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts;

or comprises (a) a self-emulsifying formulation containing picoplatin, (b) a plurality of stabilized picoplatin nanoparticles, (c) a picoplatin solid dispersion in a water-dispersible matrix material, and/or (d) a nanoparticulate picoplatin suspension in a medium chain triglyceride or a fatty ester; or any combination thereof.

In various embodiments, the invention provides unit dosage forms for oral administration of picoplatin in accord with methods of the invention.

### DETAILED DESCRIPTION

### Definitions

“Picoplatin” refers to cis-amminedichloro(2-methylpyridine)platinum(II), or [SP-4-3]-ammine(dichloro)(2-methylpyridine)platinum(H) as the drug is also termed, the structure of which is shown above. It is a compound belonging to the general class of transition metal complexes, in this case a complex of the third-row transition element platinum, the platinum being in the +2 oxidation state.

“Cancer” as the term is used herein includes malignancies, neoplasms and solid tumors of all organs, including small cell lung cancer (SCLC), colorectal cancer, ovarian cancer, and prostate cancer, including hormone-refractory prostate cancer, also termed castration-resistant prostate cancer (CRPC).

A “unit dosage form” or “dosage form” as the terms are used herein refer to a physical dosage form that is adapted for oral administration, e.g., ingestion, wherein the form provides a preselected dose per administration, adapted to provide for a complete release of the drug, which may be rapid, controlled or prolonged release, in vivo after administration of the dosage form. A dosage form can be a capsule, a tablet, or measured quantity of a liquid or dispersed formulation, or the like. The dosage form is the physical entity in which the picoplatin is administered.

The term “individual dose” as used herein refers to a quantity of picoplatin administered substantially concurrently to the patient by administration of one or more unit dosage forms adapted for oral administration. An “individual dose” can therefore include one or more individual dosage forms, provided they are administered substantially concurrently. For example, a 200 mg individual dose can be achieved by administering one, two, or more, unit dosage forms of picoplatin, of appropriate size, within a few minutes of each other.

The terms “daily dose” or “aggregate daily dose” as used herein refers to the sum of the amount of individual doses given in the course of a full day, i.e., 24 hours. A “daily dose” or an “aggregate daily dose” can comprise one or more spaced individual doses, each individual dose comprising one or more unit dosage forms of the invention. For example, a daily dose of 150 mg of picoplatin can be provided by administering three spaced individual doses, each comprising 50 mg of picoplatin, e.g. every 8 hours. For example, a daily dose of 200 mg can be provided by administering two spaced individual doses, each comprising 100 mg of picoplatin, e.g., spaced 12 hours apart, each comprising two 50 mg unit dosage forms administered substantially concurrently. Preferably the daily dose will be about 25-500 mg picoplatin, e.g., about 50-200 mg picoplatin.

The term “drug dosing interval” as used herein refers to a period of one or more days during which picoplatin is administered daily or intermittently, either alone or in conjunction with other therapies.

The term “drug intermission interval” as used herein refers to a period of one or more days during which picoplatin administration is suspended, although other forms of therapy can be continued.

The term “quasi-metronomic” as used herein refers to a schedule of dosing intervals and drug intermission intervals wherein substantially regular dosing and intermission schedules are maintained.

A “cycle” as the term is used herein with respect to a drug dosing cycle refers to a combination of a drug dosing interval and a respective drug intermission interval, when there is more than a single drug dosing interval.

Individual doses are administered “non-concurrently” when the dosage forms are administered to the patient more than about 1 hour apart. Dosage forms administered within about 1 hour of each other are considered to be administered “concurrently”, thus together constituting an individual dose.

A “maintenance dose” as the term is used herein refers to an individual dose or a daily dose of picoplatin, wherein the picoplatin is administered in individual doses of less than about 200 mg each, such that an oral bioavailability of greater than about 50% of each dose is maintained. A maintenance dose can be administered daily or on alternating days for multiple days, and can provide a steady contribution to blood plasma levels of picoplatin over time.

A “boost dose” as the term is used herein refers to a dose that contains more than about 200 mg of picoplatin administered concurrently, which can be used to rapidly increase blood plasma levels of picoplatin. A boost dose which is greater than the maintenance dose can be used in conjunction with maintenance doses to attain a target blood plasma concentration of picoplatin, which can then be attained with a schedule of maintenance doses, the maintenance doses having greater than about 50% bioavailability. A boost dose need not have greater than about 50% bioavailability, but preferably has about 30% or about 40% oral bioavailability.

“Bioavailability” and “oral bioavailability” as used herein refer to the fraction of the administered platinum from the picoplatin that reaches the systemic circulation and is not sequestered in a non-target organ or excreted without absorption via the gastrointestinal tract. The terms refer to a blood plasma concentration integrated over time as a percentage of the orally administered dose. An intravenously administered dose is defined has having an oral bioavailability of 100%. “PUF” refers to plasma ultrafiltrate.

“Daily aggregate oral bioavailability” refers to an average bioavailability in the blood stream of doses administered over the course of an entire 24 hour period.

“Administering” or “administration” refers to providing a medicinal compound to a patient in need thereof. A “frequency” of administration refers to how often the medication is given when repeated doses are prescribed; for example, the medication can be administered daily. A “duration” refers to the period of time over which repeated doses are administered; for example, the picoplatin can be administered for a duration of two weeks.

In various embodiments of the invention, a type of dosage form for oral administration according to a method of the invention, an “encapsulated form,” can comprise a solid particulate formulation including less than about 200 mg of picoplatin, and including various suitable excipients, are contained within a substantially water-soluble shell, typically a gelatin or hydroxypropyl methyl cellulose (HPMC) capsule, which contains the formulation during storage and oral ingestion. See, for example, Application No. PCT/US2008/001746, filed Feb. 8, 2008, published as WO2008/097658, entitled “Encapsulated Picoplatin”.

By “substantially water-soluble” is meant that the capsule shell is sufficiently water-soluble to allow the shell to dissolve or rupture in the gastro-intestinal (GI) tract of the mammal, so that the active ingredient of the formulation, picoplatin, can be absorbed into the mammal's bloodstream through the mucosa of the GI tract. Thus, dissolution of the substantially water-soluble shell takes place within the period of time of a typical residence of an ingested substance within the GI tract, for example, within a period of time of several hours, preferably within a period of time of less than about 30 minutes, more preferably within a few minutes after ingestion of the capsule by the patient. However, in various embodiments the tablet can be further coated, such as with an enteric coating, or otherwise designed to permit controlled or prolonged release of the picoplatin if desired.

In various embodiments, an encapsulated oral dosage form comprises a powder that comprises picoplatin, the picoplatin in physical form being a particulate of less than about 10 microns average particle size, the formulation further including a carbohydrate and a lubricant, as the terms are defined herein. The formulation may also include other ingredients, such as a dispersant/disintegrant, an antioxidant, a buffer, a colorant, and the like. The formulation is enclosed by the capsule shell to provide an encapsulated unit dosage form of the invention.

A “carbohydrate” as the term is used herein includes carbohydrates useful as fillers or as bulking agents in pharmaceutical compositions, including a monomeric, dimeric, oligomeric or polymeric sugar derivative, such as glucose, fructose, lactose, sucrose, ribose, hemicelluloses, celluloses, modified celluloses (cellulose ethers, etc.), and the like. A carbohydrate molecule comprises carbon, hydrogen and oxygen, in an approximate molar ratio of 1:2:1. However, molecules deviating from this formula, such as deoxysugars and their oligomers/polymers, are also included within the term “carbohydrate” as used herein, provided sufficient hydroxyl groups are present to confer water-solubility or water-absorbability upon the substance. A carbohydrate may also contain other elements such as nitrogen (e.g., aminosugars), sulfur (e.g., sugar sulfonic acids), and phosphorus (e.g., sugar phosphates), without departing from the principles of the invention.

By a “substantially water-soluble” carbohydrate is meant that the carbohydrate is sufficiently water-soluble to allow it to dissolve in the aqueous environment of the gastrointestinal (GI) tract within a few hours, preferably within a few minutes. An example of a substantially water-soluble carbohydrate is a monosaccharide, for example glucose.

By a “substantially water-dispersible carbohydrate” is meant a carbohydrate that, while it may not totally dissolve in water, is nevertheless of sufficient hydrophilic nature that it freely disperses in water.

By a “substantially water-absorbing” carbohydrate is meant that the carbohydrate, although it does not completely or even to any significant degree dissolve in water, it nevertheless takes up, adsorbs, or absorbs water within its physical structure. For example, cellulose, such as microcrystalline cellulose, does not dissolve in water, but it becomes hydrated in the presence of water, absorbing several times its weight in water. This absorption of water by, for example, cellulose, can assist in the dissolution of the picoplatin; it is believed that this absorption of water by the water-absorbing carbohydrate acts to assist in the dissolution of the picoplatin within the GI tract, by holding water molecules within close physical proximity to the surfaces of the finely particulate picoplatin.

By a “substantially dry” material is meant a solid substance to which no exogenous water has been added and which has a relatively low wt % of contained water, typically less than about 5 wt %, preferably less than about 1-3 wt % of water, more preferably less than about 1 wt % of water. A substantially dry material need not be absolutely anhydrous within the meaning assigned herein, but the amount of residual water present in the material is limited. For example, lactose monohydrate, which includes 5 wt % water, can be used as a carbohydrate in the dosage form.

By a “powder” is meant a material in the physical form of a solid that is divided into relatively fine particles. A powder can be a milled powder. Such powders can be made by grinding coarser powders to the desired fineness. A preferred method of forming a micronized powder is by jet milling. The powder material that is encapsulated contains the picoplatin particulate, a fine powder of less than 10 microns average particle diameter, in combination with, or incorporated within, coarser powders such as carbohydrates, which can be of sufficient fineness to pass a 20-mesh or a 30-mesh screen, but which need not be of less than 10 microns average particle diameter.

By a “particulate,” in the context of the physical form of solid picoplatin disclosed herein, is meant a very fine powder wherein the average picoplatin particle diameter is less than 10 microns, preferably less than 7 microns, most preferably wherein at least about 90% of a sample of the particulate material is composed of individual particles each having a diameter of less than about 5 microns. The finely particulate nature of the picoplatin aids in its rapid and complete dissolution in the patient's GI tract. The picoplatin particulate can be a micronized material, a microcrystalline material, a lyophilized material, or any combination thereof.

A “micronized” material is a particulate wherein the majority of the particles making up the powder have a particle diameter of about 10 microns or less. Preferably, the average particle diameter is about 5 microns or less. Particle diameters can range down to about 1 micron or less without departing from the principles of the invention. A micronized solid can be crystalline or amorphous.

A “microcrystalline” material is a fine particulate wherein the solid is in crystalline form, the crystals being predominantly of the specified dimensions. A microcrystalline material can be prepared by precipitation of the material from a solvent, such as by addition of a second liquid material in which the material is insoluble.

A “lyophilized” material is a solid that has been obtained by a step of lyophilization of a solution of the material. Lyophilization, as is well known, involves the vacuum sublimation of a solvent such as water, or other compatible solvent(s), from a frozen solution of the material, such that once the solvent is completely removed, a finely powdered solid material remains.

By the term “cellulose” is meant herein a polymeric carbohydrate material made up mostly of a linear polymer of β(1-4)-linked D-glucose units. Cellulose is typically derived from a natural source such as wood pulp, cotton, or bacteria. Cellulose may be ground or comminuted to create a finely particulate material. Alternatively, microcrystalline cellulose, such as is sold under the trademark Avicel®, can be used. For example, the Avicel® can be Avicel PH101®. By microcrystalline cellulose is meant a cellulose which has been subjected to partial acid hydrolysis, which serves to predominantly hydrolyze the amorphous regions of a sample of cellulose, leaving the more crystalline domains intact. Microcrystalline cellulose takes the physical form of a fine powder.

The term “modified cellulose” as used herein refers to a chemically or biologically modified cellulose. For example, sodium carboxymethyl cellulose, that is, cellulose that bears pendant carboxymethyl groups as sodium salts, as is well known in the art, is a modified cellulose within the meaning herein. Likewise, methyl cellulose, ethyl cellulose, hydroxyethyl cellulose, hydroxypropyl cellulose, and hydroxypropyl methyl cellulose (HPMC) are modified celluloses within the meaning assigned herein. A cross-linked sodium carboxymethyl cellulose, also known as “croscarmellose sodium,” is a cross-linked modified cellulose within the meaning herein. Croscarmellose sodium is a dispersant/disintegrant within the meaning of the term herein.

A “lubricant” or “glidant” within the meaning herein is a substance that serves to coat the surface of particles and reduce the friction of inter-particle movement, such as during powder handling operations, for example, when filling capsules. Reducing the friction serves to reduce static electricity buildup and particle clumping or aggregation, for example during the milling, powder handling, and capsule filling processes typically used to produce the encapsulated unit dosage form of the invention or an encapsulated unit dosage form produced by the method of the invention.

A “dispersant” or “disintegrant” is a substance that is a component of a formulation of the invention that aids in the dispersion of the finely particulate formulation of the invention upon exposure to an aqueous medium, for example within the GI tract of a patient to whom the unit dosage form of the invention has been administered. It is believed that dispersants act to increase the solvation of the surfaces of solid particles within the aqueous medium, thereby reducing particle-particle adhesion and clumping while aiding in dissolution of the solid through improved surface wetting. Examples of dispersant include croscarmellose sodium and povidone. Povidone, also known as poly(vinylpyrrolidone), is a polymeric material bearing multiple pyrrolidone units along a poly(vinyl) backbone.

In various other embodiments, a “tablet” oral dosage form that can be used to practice a method of the invention refers an oral dosage form including a core and, optionally, a coating. The core includes a solid body of substantially dry powder including the picoplatin particulate of less than about 10 microns average particle diameter, a filler, and a lubricant. See, for example, Application No. PCT/US2008/001752, filed Feb. 8, 2008, published as WO2008/097661, entitled “Stabilized Picoplatin Oral Dosage Form”.

The core can be formed by compaction, molding, or can be a granulated material. The average particle diameter of the picoplatin particulate can also be less than about 7 microns. The distribution of average particle diameters in the picoplatin particulate can be such that about 90% of the particles have individual particle diameters of less than about 5 microns. By “average particle diameter” is meant a number average particle diameter, as is well known in the art. The core is surrounded by a coating that covers the core, and serves to contain the materials of the core during storage and oral ingestion, as well as to protect the picoplatin contained in the core from various degradative agents such as light and oxygen. The core and the coating(s) (“the tablet dosage form”) preferably do not contain any substantial amounts of redox-active metal salts such as transition metal salts. By a “granulate” is meant a divided form of a solid material formed of a plurality of individual granules of an intermediate coarseness, less fine than a powder, but not a monolith.

The tablet oral dosage form is substantially water soluble, being adapted for oral administration. By “substantially water-soluble” is meant that the dosage form is sufficiently water-soluble to allow it to dissolve or disperse within the gastro-intestinal (GI) tract of the patient, so that the active ingredient of the formulation, picoplatin, can be absorbed into the patient's bloodstream through the mucosa of the GI tract. Thus, dissolution takes place within the period of time of a typical residence of an ingested substance within the GI tract, for example, within a period of time of several hours, preferably within a period of time of less than about 30 minutes, more preferably within a few minutes after ingestion of the tablet by the patient. However, although rapid dissolution is usually preferred, the tablet dosage form can be further coated or otherwise adapted to permit controlled or prolonged release of the picoplatin, if desired.

The tablet dosage form can be a coated tablet. By a “coated tablet” is meant herein a dosage form with a compacted powder core and a coherent coating, either hard or soft in texture, covering the core. The coating can be a plastic material. A “plastic” material, as the term is used herein, is a relatively malleable solid material that has sufficient rigidity to maintain a shape once formed, but which can be molded under pressure, for example, soft gelatin. The coating can be a water-soluble or water-dispersible substance that can be molded under pressure or applied as a viscous solution then subsequently dried, for example gelatin, a synthetic polymer, for example polyvinyl alcohol, or semi-synthetic polymer, for example hydroxypropyl methyl cellulose. The coating can be a layer of a water soluble solid such as a sugar, for example sucrose, that forms a sufficiently viscous solution to allow coating of the core with the viscous solution followed by drying of the coating that is thus applied.

The dosage form can be a geltab. A “geltab” as the term is used herein refers to the dosage form comprising a coating, which can be soft gelatin or another soft, pliable gel-like material surrounding the compacted powder core. Such a coating is also preferably free of redox-active metal salts.

The dosage form can be a coated pill. A “pill” as the term is used herein is a molded but not compressed core wherein a binder assists in holding the picoplatin particulate and other components in a coherent mass. The dosage form can also be a sachet, wherein particulates are granulated and the granulations are coated individually or in small numbers, wherein pluralities of the particulates can be enclosed in packaging and then administered to provide the total dosage.

A “light-attenuating” coating, as the term is used herein, refers to a coating layer covering the tablet that is adapted or treated so as to attenuate the intensity of light transmitted by the coating to the core containing the picoplatin. A coating may be light-attenuating without completely blocking or reflecting all incident light within the meaning herein. An “opaque” coating blocks or reflects most incident light. An “opaquifying” (or “opacifying”) agent is a material or a structure that serves to attenuate, reflect, disperse or absorb incident light such that the intensity of the light passing through the material containing the opaquifying agent is reduced compared to the intensity of the incident light. A light-attenuating coating is desirable due to the possible photo-decomposition of picoplatin, even in the solid form. An example of a light-attenuating coating is a coating comprising calcium sulfate, for example, a coating formed of a plasticized hydroxylpropyl methyl cellulose such as OPADRY® containing dispersed, solid calcium sulfate. Other salts, such as magnesium sulfate, can be used, provided that no redox-active metal salts such as transition metal salts are included.

A “core” as the term is used herein refers to a solid body of a compacted, molded, or compressed powder that can be derived from a coarser granulate that can be compacted in the final dosage form, or can be molded, or can be used as a granulate that comprises picoplatin as a particulate of less than about 10 microns average particle size. The core further includes a filler comprising a carbohydrate and a lubricant, as the terms are defined herein. The core may also include other ingredients, such as a dispersant/disintegrant, an antioxidant, a buffer, a colorant, and the like. The core is preferably free of any redox-active metals or metal salts.

The core is covered by the coating, which is free of redox-active metal salts, to provide the tablet oral dosage form of the invention. A “coating” as the term is used herein refers to a water-soluble or water-dispersible solid that is suitable for covering and sealing the core. Examples, such as are discussed above, include the coatings of a coated tablet, pill, granulation, or geltab. The coating can include ingredients such as an opaquifying material, for example calcium sulfate, an antioxidant, a colorant, a flavorant, and the like. The coating can also include imprints or embossed characters such as letters, numbers or symbols that are visually apparent and convey useful information to a care provider or a patient, such as the amount of the active ingredient picoplatin in the dosage form. There can also be a second coating on the outer surface of the first coating. The second coating is also preferably free of redox-active metal salts.

A “redox-active metal salt” as the term is used herein refers to salts of metals that can enter into redox reactions with picoplatin and includes transition metal salts such as Fe⁺³, but excludes the salts of Group 1 and Group 2 metals, i.e., alkali and alkaline earth metals such as Na, K, Mg, Ca, and the like.

A “transition metal salt” as the term is used herein, refers to salts of transition metals such as titanium, iron, copper, zinc and the like. The term does not encompass salts of aluminum or silicon. The term specifically includes oxides of transition metals, such as titanium oxide and iron oxide. It is recognized that picoplatin itself is generally not understood to be a transition metal salt, and, as the active pharmaceutical ingredient of the oral dosage form of the invention, picoplatin is not excluded from the dosage form. The terms “transition metal salt” and “redox-active metal salt” as used herein specifically exclude picoplatin and/or Pt-containing manufacturing impurities or degradation products derived from picoplatin. “Substantially free of a redox-active metal salt” means that the coating and/or core do not contain an amount of a redox-active metal salt, for example a transition metal salt, that can degrade the picoplatin, e.g., can reduce its bioactivity.

By “substantially free” of a redox-active metal salt or transition metal salt is meant that the coating or core has levels of one or more of these salts that do not, singly or in combination, significantly contribute to the degradation of the picoplatin, either in vitro e.g., in storage, or in vivo, e.g., after ingestion. Usually such amounts are no more than the total of the trace amounts of such salts normally present in adjuvants formulated or prepared so as to exclude them entirely.

A “compacted” powder as can form the core of the tablet dosage form is a powder that has been subjected to sufficient pressure to compress the powder, thereby removing air or optionally an inert gas from between the individual powder particles and causing the particles to fuse or adhere to each other. The particles adhere to each other with sufficient strength to allow at least the limited amount of handling needed to subsequently apply the coating material. A binder may be present in the powder to assist in the particles adhering to each other in the formation of the core. Alternatively a carbohydrate of the filler may act as a binder. Upon dissolution of the dosage form, for example in the GI tract of a patient, the particles disperse and dissolve.

A “molded” powder as can form the core of the dosage form is a powder that has been assembled into a cohesive mass without compression, such as by use of a binder that serves to cause adhesion of the powder. A “granulation” as can form the core of the dosage form is a particulate of relatively small size wherein each particulate can be covered with the coating to provide a plurality of coated granulated particles, such as forms a sachet.

A “filler” as the term is used herein refers to a water-soluble or water-dispersible solid composition comprising a carbohydrate. The core of the tablet oral dosage form can include about 40-80 wt % of a filler. The filler serves to disperse the particulate picoplatin, inhibiting clumping of the sub-10 micron picoplatin particles, stabilizing the picoplatin chemically, and assisting in the dispersion or dissolution of the picoplatin in an aqueous medium. The bulk of the core in addition to the picoplatin, the lubricant, and the dispersant, if any, is generally provided by the filler, although additional ingredients can be present in the core.

In various other embodiments, a liquid or dispersed oral dosage form that can be used to practice a method of the invention can comprise (a) a self-emulsifying formulation containing picoplatin, (b) a plurality of stabilized picoplatin nanoparticles, (c) a picoplatin solid dispersion in a water-dispersible matrix material, (d) a nanoparticulate picoplatin suspension in a medium chain triglyceride or a fatty ester, or any combination thereof. See for example, Application No. PCT/US2008/008669, filed Jul. 16, 2008, published as WO 2009/011861, entitled “Oral Formulations for Picoplatin”.

“Self-emulsifying” refers to a property of a formulation wherein upon contacting the formulation with an aqueous medium, such as in the gastro-intestinal tract of a patient, the formulation spontaneously forms an emulsion.

“Nanoparticles” are solid particles of an average particle diameter of less than about 1 micron (micrometer, μm). One micron is 1,000 nanometers (nm).

“Stabilized” nanoparticles are picoplatin nanoparticles coated with a stabilizing material and having a reduced tendency for aggregation and loss of dispersion with respect to nanoparticles of picoplatin without a stabilizing coating.

“Casein” is a milk-derived protein that typically is globular in aqueous dispersion, as is well known in the art. A “caseinate” is a salt form of casein wherein carboxylate groups in the protein are present in ionized form, such as the sodium salts (“sodium caseinate”).

“Microfluidization” is a technique for preparing dispersions of fine particles in a liquid medium wherein coarser particles are comminuted in the presence of the liquid medium.

“High-shear mixing” is a technique for preparing dispersions of fine particles in a liquid medium wherein high-shear conditions comminute coarser particles into finer ones in the presence of the liquid medium.

A “solid dispersion” as the term is used herein refers to a dispersion of solid picoplatin in a solid or semi-solid matrix. The solid dispersion can be formed in a liquid or melt phase wherein the final mixture solidifies into the solid or semi-solid form.

“Water-dispersible” means that a solid or semi-solid material can be suspended in an aqueous medium and does not spontaneously phase separate from the aqueous medium. “Water-dispersible” includes “water-soluble”, referring to a solid or semi-solid material that completely dissolves in the aqueous medium to form a homogeneous solution. A “matrix” as the term is used herein refers to an organic material, that is at least dispersible in water, that is solid at about room temperature or about human body temperature, in which picoplatin can be dispersed.

An “oil” as the term is used herein refers to an organic liquid, which is water-insoluble, or at least only partially water-soluble, that can form a separate phase in the presence of water. An example of an “oil” is a glyceride such as a medium chain triglyceride, or a medium chain mono- or di-glyceride, or castor oil. Another example of an oil is a fatty ester. A fatty ester refers to an alkyl ester of a fatty acid. An example is ethyl oleate. “MCT oil” refers to medium chain triglyceride oil. Examples include the MCT oil sold under the Miglyol trademark, such as Miglyol 912, a caprylate/caprate (octanoate/decanoate triglyceride).

A “nanodispersion” is a dispersion of picoplatin particles of less than 1 μm average particle diameter in a liquid, for example in MCT oil or in a fatty ester.

A “lecithin” as the term is used herein is a mixture of triglycerides, glycolipids, and phospholipids such as phosphatidylcholine, as is well-known in the art. Lecithins can be derived from eggs or from soy beans. A high-phosphatidylcholine lecithin is a lecithin with a relatively high phosphatidyl-choline (PC) content. A low-phosphatidylcholine lecithin is accordingly a lecithin with a relatively low PC content.

A “surfactant” as the term is used herein is a substance that reduces interfacial surface tension between immiscible liquids such as oil and water, reduces surface tension of a water drop, and exhibits other surface-active properties as are well known in the art.

The term “weight average molecular weight” is well known in the art and characterizes an average molecular weight of a polydisperse sample of a polymer.

A “PEG” or a “polyethyleneglycol” is a polymeric material composed of repeating —CH₂CH₂O— units, wherein there are two or more units. Thus, diethyleneglycol and all higher polymers are polyethyleneglycols within the meaning herein. A polyethyleneglycol can have a free OH group at either terminus or at both termini, or can alternatively include other groups such as an alkoxy or aryloxy group at one or both ends, for example a methoxy group as in a polyglyme CH₃O—(CH₂CH₂O)ₙ—OCH₃. Such an ether-terminated PEG can also be referred to as a “polyethyleneglycol ether”. PEG-400 is a PEG with a weight average molecular weight of about 400 DA. PEG-8000 is a PEG with a weight average molecular weight of about 8000 DA. A compound can be “PEG-ylated”, meaning that it bears at least one PEG group, which can be introduced in a variety of ways, such as by polymerization of ethylene glycol initiated by the compound, or coupling of the compound with a preformed PEG. For example, Gelucire® is a PEG-ylated fatty acid monoglyceride, meaning that a glycerol moiety bears a single fatty acid moiety and PEG moieties on one or both of the remaining free hydroxyl groups.

A “dipolar aprotic solvent” is a solvent not containing a source of protons in aqueous solution (an example of a protic solvent is ethanol) that also is polar in character and is typically at least partially soluble in water. Examples of aprotic solvents are DMF, NMP, DMSO, DMAC, and the like. “DMSO” is dimethylsulfoxide. “NMP” is N-methylpyrrolidone. “DMF” is N,N-dimethyl-formamide. “DMAC” is N,N-dimethylacetamide.

“Labrasol®” is a mixture composed of about 30% mono-, di-, and triglycerides of C8 and C10 fatty acids, 50% of mono- and di-esters of polyethyleneglycol (PEG 400), and 20% of free PEG 400. Labrasol® has surfactant properties.

“Cremophor RH 40®” is a nonionic solubilizer and emulsifying agent obtained by reacting 45 moles of ethylene oxide with 1 mole of hydrogenated castor oil. The main constituent of Cremphor RH 40® is glycerol polyethylene glycol oxystearate, which, together with fatty acid glycerol polyglycol esters, forms the hydrophobic part of the product. The hydrophilic part consists of polyethylene glycols and glycerol ethoxylate.

“Cremophor ELP®” is a nonionic solubilizer made by reacting castor oil with ethylene oxide in a molar ratio of 1:35.

“Gelucire®” including Gelucire 44/14 (CAS RN 121548-04-7) and Gelucire 50/13 (CAS RN 121548-05-8) are fatty acid glycerides bearing polyethyleneglycol (PEG) groups. For example, Gelucire 44/14 is a PEG-ylated glyceride of lauric acid; Gelucire 50/13 is a PEG-ylated glyceride of stearic acid. The numbers after the word Gelucire refer to the melting point in ° C. and the hydrophilic-lipophilic balance (HLB) value respectively. Gelucire compounds are PEG-ylated with PEG 1500 (polyethyleneglycol of weight average molecular weight 1500 DA).

“Polysorbate 80” refers to sorbitan mono-9-octadecanoate poly(oxy-1,2-ethanediyl) derivatives; they are well known as complex mixtures of polyoxyethylene ethers used as emulsifiers or dispersing agents in pharmaceuticals.

“Phospholipon 90G” or “PL90G” (American Lecithin Products, Oxford, CT) is a tradename for lecithin, minimum 94% phosphatidylcholine for the manufacture of liposomes. “Phospholipon 90H” or “PL90H” is a hydrogenated PL90G. The term “PL90” refers to either one of these materials.

“Vitamin E TPGS” refers to the compound D-alpha-tocopheryl polyethylene glycol 1000 succinate.

“Compritol 888” refers to glyceryl behenate. A “behenate” is an ester of docosanoic acid, as is well known in the art.

“Poloxamer 188” (CAS RN 9003-11-6) is a Polyethylene-Polypropylene Glycol copolymer of the formula HO(C₂H₄O)ₐ(C₃H₆O)_{b}(C₂H₄O)ₐH with a weight average molecular weight of about 8400

“SPAN 60” refers to sorbitan monostearate.

“Kollidon K90” (Hoechst, Germany) refers to a polyvinylpyrrolidone with a molecular weight of about 90,000.

“Miglyol 812” (Sasol Germany GmbH, Witten, Germany) refers to a medium chain triglyceride wherein the acid moieties are caprylic and capric acid. Miglyol is a trademark identifying the source of this and other varieties of MCT oil.

A “second medicament comprising an anticancer medicament” can include, without limitation, a taxane (e.g.: paclitaxel (Taxol®) or docetaxel (Taxotere®)), a tyrosine kinase and/or a growth factor receptor inhibitor such as a VEGFR inhibitor (e.g.: monoclonal antibodies such as: bevacizumab (Avastin®), trastuzumab (Herceptin®), panitumumab (Vectibix®) or cetuximab (Erbitux®)); a cephalotaxine analog (e.g.: topotecan (Hycamtin®); irinotecan; 9-aminocamptothecin; Rubitecan®; Exatecan®; XR-5000, XR-11576); an anti-metabolite (e.g.: capecitabine (Xeloda), gemcitabine, 5-FU with or without leucovorin, S1 (gimeracil/oteracil/tegafur), tegafur/uracil, methotrexate, or a thymidylate synthease inhibitor (Tomudex®, ZA9331, LY231514 (pemetrexed))); a protein kinase inhibitor (e.g.: sorafenib (Nexavar®), dasatinib (Sprycel®), gefitnib (ZD1839, Iressa®), imatinib (Gleevac®), lapatinib (Tykerb®), cediranib, also known as AZD2171 (Recentin®), erlotinib (Tarceva®) or sunitinib (Sutent®)); an anthracyclin (e.g.: amrubicin, doxorubicin, liposomal doxorubicin, epirubicin, idarubicin, pegylated doxorubicin or pegylated liposomal doxorubicin (Doxil®)); a Vinca alkaloid (e.g.: vinorelbine (Navelbine), vincristine, vinblastine, vindesine); a podophyllotoxin analog (e.g.: etoposide, teniposide); a growth factor inhibitor (e.g.: inhibitor of PDGF, endothelial GF, VEGF, EGF, or hepatocyte GF; for example an GF-binding antibody or a GF receptor-binding antibody); an inhibitor of cell cycle kinases (such as CDK-2, CDK-4, or CDK-6); a cytostatic agent (Tamoxifen, Toremifene, Raloxifene, Droloxifene, Iodoxyfene; megestrol acetate; an aromatase inhibitor such as Anastrozole (ZD1033), Letrazole, Vorazole, Exemestane; an antiandrogen such as Flutamide, Nulutamide, Bicalutamide, Cyproterone acetate; an LHRH agonist or antagonist such as Foserelin acetate or Luprolide; an inhibitor of testosterone dihyhdroreductase such as Finasetide, a metalloproteinase inhibitor such as Marimastat or a uPAR inhibitor); an alkylating agent (e.g.: melphalan, cyclophosphamide, ifosphamide, nitrosourea, carmustine, lomustine); or radiation therapy (e.g.: X-ray, γ-ray, particle beam, brachytherapy, radioisotope).

Alternatively, the additional medicament is a non-platinum containing agent, can be selected to treat a complication of the cancer, or to provide relief to a subject from at least one symptom of the cancer, for example, sirolimus or rapamycin (Rapamune®), dexamethasone (Decadron®), palonosetron HCl (Aloxi®), aprepitant (Emend), ondansetron (Zofran), or granisetron (Kytril®).

Orally active anticancer agents that can be administered include altretamine (Hexalen®), an alkylating agent; capecitabine (Xeloda®), an anti-metabolite; dasatinib (Sprycel®), a TK inhibitor; erlotinib (Tarceva®), an EGF receptor antagonist; gefitinib (Iressa®), an EGF inhibitor; imatinib (Gleevec®), a TK inhibitor; lapatinib (Tykerb®), an EGFR inhibitor; lenalidomide (Revlimid®), a TNF antagonist; sunitinib (Sutenn, a TK inhibitor; S-1 (gimeracil/oteracil/tegafur), an anti-metabolite; sorafenib (Nexavar®), an angiogenesis inhibitor; tegafur/uracil (UFT®, Uftoral®), an anti-metabolite; temozolomide (Temodar®), an alkylating agent; thalidomide (Thalomid®), an angiogenesis inhibitor; topotecan (Hycamtin® for injection or Oral Hycamtin®), vinorelbine (Navelbine®), an anti-mitotic; cediranib (AZD2171, Recentin®), a VEGF inhibitor; and/or vorinostat (Zolinza®), a histone deacetylase inhibitor.

As the term is used herein, “radiation” or “radiotherapy” refers to the treatment of cancer patients with various forms of ionizing radiation, which acts to a great extent on dividing cells by interfering with DNA replication and cell division. The three main types of radiotherapy are external beam radiotherapy (EBRT or XBRT) or teletherapy, brachytherapy or sealed source radiotherapy and unsealed source radiotherapy. The differences relate to the position of the radiation source; external is outside the body, while sealed and unsealed source radiotherapy has radioactive material delivered internally. External beam radiotherapy can involve beams of photons, such as X-rays, or beams of particles, such as protons. External beam radiotherapy can involve either total body irradiation or the use of multiple focussed beams to concentrate the energy in a defined volume of body tissue. Brachytherapy involves implantation of sealed sources of various radioisotopes within body tissues, such that the sources can be removed after a period of time. The type of radiation emitted depends on the identity of the radioisotope included in the sealed source, and can be photon (X-ray) or particle (e.g., beta particle). When unsealed sources are used, e.g., radiolabeled antibodies or the like, the nature of the radiation again depends on the identity of the radioisotope used, but due to the fact that there is no containment, particles of shorter range such as alpha particle and Auger electrons can be used effectively. However, since unsealed sources typically cannot be removed surgically, the radioisotopic form must be one that can be excreted, or else decays, within an appropriate time frame. Examples of useful isotopes include ⁹⁰Y, ¹³¹I, and ¹⁷⁷Lu.

### Description

The aggregate daily dose is provided by oral administration of one or more individual doses of the picoplatin. Each individual dose is less than about 200 mg. The oral administration can be carried out using oral dosage forms that can include capsules, tablets, and liquid and dispersion formulations such as described herein.

Capsules suitable to deliver picoplatin orally include those disclosed in PCT/US2008/001746, published as WO2008/097658, which is incorporated by reference herein.

Tablets suitable to deliver picoplatin orally include those disclosed in PCT/US2008/001752, published as WO2008/097661, which is incorporated by reference herein.

Liquid or dispersion oral dosage forms suitable to deliver picoplatin orally include those disclosed in PCT/US2008/008669, published as WO2009/011861, which is incorporated by reference herein.

It has unexpectedly been found that the oral bioavailability of smaller amounts of picoplatin, less than about 200 mg per individual dose, whether given as one or a plurality of unit dosage forms, is much higher than the oral bioavailability of individual doses containing 200 mg or more of picoplatin. For example, the present regimen provides oral bioavailability of the picoplatin in the patient after ingestion that is greater than about 50%. In various embodiments, the oral bioavailability can be greater than about 75%, or greater than about 90%. For example, as shown below in Table 3, oral bioavailability of an individual dose of 50 mg is greater than oral bioavailability of an individual dose of 100 mg, which is greater than oral bioavailability of an individual dose of 200 mg, which is greater than oral bioavailability of an individual dose of 300 mg or 400 mg.

This discovery provides for the invention of a method to administer uniform, preselected amounts of picoplatin to achieved preselected plasma levels. These individual doses can be more completely absorbed into the blood stream than when dosing with amounts of picoplatin that are less bioavailable and so are excreted without therapeutic effect, or absorbed into non-target tissues or organs.

In various embodiments, the invention provides a method of treating cancer in a human patient afflicted therewith, comprising administering orally to the patient one or more individual doses per day each comprising picoplatin, wherein each individual dose comprises less than about 200 mg of picoplatin, wherein an aggregate daily dose comprises a sum of the one or more individual doses administered within a single day, provided that when more than one individual dose makes up an aggregate daily dose, each individual dose is administered non-concurrently with each other individual dose over the course of the day. In various embodiments, an oral bioavailability of each individual dose is greater than about 50%, or, a daily average oral bioavailability of the picoplatin to the patient from the daily aggregate dose is greater than about 50%, or both.

In various embodiments, the method of the invention can further comprise administration of the individual doses, each comprising less than about 200 mg of picoplatin, throughout a drug dosing interval comprising a successive or intermittent plurality of days. An intermittent plurality of days can comprise administration every other day, or other regular or irregular schedules. In various embodiments, the method can further comprise quasi-metronomic dosing comprising administering the picoplatin to the patient throughout a plurality of drug administration cycles comprising a duration of treatment, each cycle comprising administering the individual doses, each comprising less than about 200 mg of picoplatin, throughout a drug dosing interval comprising one or more days, followed by a respective drug intermission interval comprising one or more days.

In various embodiments, the method comprises one or more drug dosing intervals wherein in at least one of the drug dosing intervals further comprises administration of a boost dose, the boost dose comprising 200, 300, 400, or 500 mg or incremental quantities therebetween of picoplatin.

In various embodiments, the invention provides a method wherein administration of an individual dose of less than 200 mg comprises administration of one or more encapsulated dosage forms comprising a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant.

In various embodiments, the invention provides a method wherein administration of an individual dose of less than 200 mg comprises administration of one or more tablet dosages forms comprising a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts.

In various embodiments, the invention provides a method wherein administration of an individual dose of less than 200 mg comprises administration of one or more liquid or dispersed dosage forms comprising (a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester, or any combination thereof.

In various embodiments, the aggregate daily dose can comprise a plurality of spaced individual doses of picoplatin. For example, each aggregate daily dose can be provided by spaced administration of two individual doses each comprising less than about 200 mg of picoplatin apiece for a daily dose of 400 mg, or less than about 100 mg of picoplatin apiece for a daily dose of 200 mg picoplatin. More specifically, the doses can be regularly spaced, i.e., every 12 hours. Or, each aggregate daily dose can be provided by spaced administration of three individual doses each comprising less than about 200 mg of picoplatin apiece. In this schedule also, the doses can be regularly spaced.

In various embodiments, when more than one individual dose per day is administered, the individual doses can be administered about 2-23 hours apart.

In various embodiments, a dosing interval of about 1-30 days can be followed by a drug intermission interval of about 1-4 weeks. Or, a dosing interval of about 1-5 days can be followed by a drug intermission interval of about 3 weeks. Dosing intervals and intermission intervals can be selected based upon the best judgment of the physician in attendance, based upon his or her knowledge and experience and informed by the response of the particular patient being treated.

In various embodiments, the method can comprise a plurality of cycles of a dosing interval followed by a respective drug intermission interval. For example, 2 to about 10 cycles can be used, or about 4-6 cycles can be used. Alternatively, the treatment cycles can continue until remission of the cancer or death of the patient. In various embodiments, the duration of treatment can be about 2 weeks to about 40 weeks ,or can be about 10 weeks to about 30 weeks, depending upon the judgment of the physician in attendance.

It may be viewed by the attending physician as advantageous to rapidly achieve a high steady state blood plasma concentration of picoplatin, or to temporarily increase picoplatin levels. Accordingly, one or more of the dosing intervals further can comprise administration of one or more boost doses of up to 400 mg picoplatin apiece, each boost dose comprising one or more individual doses of up to 400 mg apiece. Although a boost dose may itself have a lower oral bioavailability than do maintenance doses of less than 200 mg apiece, the absolute concentration of picoplatin in blood plasma can be rapidly increased thereby.

In addition to maintaining a therapeutic steady state plasma level of picoplatin that nevertheless avoids to the greatest extent possible side-effects and toxicity, such as neutropenia, thrombocytopenia, anemia, nausea, vomiting, fatigue, neuropathy, diarrhea, leucopenia, or alopecia, which are known side-effects of organoplatinum chemotherapy, achieving a high oral bioavailability is desirable. When high oral bioavailability is achieved, less picoplatin is sequestered in non-target organs or remains in the intestine causing unwanted damage to that tissue.

In various embodiments, the oral bioavailability of the picoplatin in the patient after ingestion of each individual dose is greater than about 50%, or is greater than about 60-75%, or is greater than about 90%. In various embodiments, the daily average oral bioavailability of the picoplatin to the patient from the daily aggregate dose is greater than about 50%, or is greater than about 60-75%, or is greater than about 90%. High oral bioavailability is surprisingly found by the inventors herein to be achieved by administration of relatively small individual doses, such as individual doses of less than about 200 mg apiece. The aggregate daily dose, which can be larger, can be achieved by successive non-concurrent administration of individual doses, each individual dose being less than about 200 mg. A larger aggregate daily dose can be achieved by spaced administration of individual doses of less than about 200 mg apiece, or less than about 100 mg apiece, or less than about 50 mg of picoplatin apiece.

In various embodiments, the aggregate daily dose of picoplatin can be about 10-500 mg, or can be about 25-150 mg.

In various embodiments, each individual dose can comprise about 10-180 mg of picoplatin, or can comprise about 25-175 mg of picoplatin, or can comprise about 50-150 mg of picoplatin, or can comprise about 50-100 mg of picoplatin. Alternatively, each individual dose can comprise 50, 60, 70, 80, 90, 100, 110, 120, 130, 140, 150, 160, 170, 180, or 190 mg, or incremental quantities therebetween, of picoplatin. As shown in Table 3, below, individual doses of 50 mg, or 100 mg, achieve a high oral bioavailability than do greater doses.

Various dosing schedules can be used. In various embodiments, the successive daily doses comprise daily doses administered for a period of 2 days to 5 months, or the intermittent daily doses comprises doses administered about every other day for a period of 3 days to 5 months. Drug intermission intervals can be about 2 days to about 4 weeks, or can be about 2 to 3 weeks.

It is desirable to achieve a steady state concentration of picoplatin in the patient's blood plasma at therapeutic levels. In various embodiments, a steady state level of picoplatin at therapeutic levels in the patient's blood is attained in about 3-5 days. The slow rise of the picoplatin level to a therapeutic level, compared to the near instantaneous rise with the use of intravenous (IV) administration, even if it takes place over a period of hours, can provide a benefit to the patient in terms of avoiding harmful side effects, such as are known to generally occur with the use of organoplatinum anticancer drugs. When using IV administration, a peak level is reached very rapidly, then the serum level falls off in an exponential manner over time. Using a method of the invention, the rate at which a peak serum concentration of picoplatin is reached can be adjusted by altering the daily dose, the dosing interval(s), and the drug intermission interval(s).

In various embodiments, each individual dose can be administered after at least about 4 hours of fasting. Administration of the oral dosage forms on an empty stomach can assist in absorption of the picoplatin into the blood stream. Also, administration of each individual dose can be followed by at least about 2 hours of fasting, to facilitate retention of the picoplatin undiluted in the digestive system until maximum absorption has occurred. For example, the one or more individual doses can be administered during the night time, when patients are not as inclined to expect a meal.

In various embodiments, the drug dosing interval and the drug intermission interval can each be adjusted in duration based upon an evaluation of severity of picoplatin side-effects in the patient following a first or subsequent administration of picoplatin. The knowledge and experience of the attending physician can guide the selection of appropriate quasi-metronomic dosing schedules for each particular patient. Likewise, an aggregate daily dose of picoplatin can be adjusted based upon an evaluation of severity of picoplatin side-effects in the patient following a first or subsequent administration of picoplatin. Such side-effects can include neutropenia, thrombocytopenia, anemia, nausea, vomiting, fatigue, neuropathy, diarrhea, leucopenia, or alopecia, or any combination thereof.

In various embodiments, a method of the invention can also further comprise administering at least one non-platinum anti-cancer agent to the human sequentially or concurrently with the picoplatin. For example, the non-platinum anti-cancer agent can comprise taxotere, taxol, irinotecan, or other agents as disclosed herein. See, for example, Table 1, below.

In various embodiments, a method of the invention can further comprise treatment with ionizing radiation. For example, the ionizing radiation can be X-ray, gamma rays, proton beam, meson beam, or radioisotope radiation from external or implanted source.

In various embodiments, the invention provides a unit dosage form of picoplatin for oral administration for use in a method of the invention. For example, a unit dosage form of picoplatin of the invention can comprise about 0.1 mg to less than about 200 mg of picoplatin.

In various embodiments, the invention comprises a dosage form adapted for the boost dose of the invention, comprising 200 mg or more of picoplatin.

More specifically, an encapsulated unit dosage form of the invention can comprise a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant.

Alternatively, a tablet unit dosage form of the invention can comprise a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts.

Alternatively, a liquid or dispersed oral formulation unit dosage form of the invention can comprise (a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester, or any combination thereof.

In any of the dosage forms, each dosage form can comprise about 25-150 mg of picoplatin, or can comprises about 50-100 mg of picoplatin.

In various embodiments, the invention provides a use of picoplatin for treatment of cancer, the use comprising administering orally to the patient one or more individual doses per day each comprising picoplatin, wherein each individual dose comprises less than about 200 mg of picoplatin, wherein an aggregate daily dose comprises sum of the one or more individual doses administered within a single day, provided that when more than one individual dose makes up an aggregate daily dose, each individual dose is administered non-concurrently with each other individual dose over the course of the day, wherein an oral bioavailability of each individual dose is greater than about 50%.

In various embodiments, the invention provides a method of treating cancer in a human patient afflicted therewith, comprising administering orally to the patient one or more individual doses per day each comprising picoplatin, wherein each individual dose comprises less than about 200 mg of picoplatin, wherein an aggregate daily dose comprises sum of the one or more individual doses administered within a single day, provided that when more than one individual dose makes up an aggregate daily dose, each individual dose is administered non-concurrently with each other individual dose over the course of the day; wherein an oral bioavailability of each individual dose is greater than about 50%, or wherein a daily average oral bioavailability of the picoplatin to the patient from the daily aggregate dose is greater than about 50%;

the method further comprising administration of the individual doses throughout a drug dosing interval comprising a successive or intermittent plurality of days; the method further comprising administering the picoplatin to the patient throughout a plurality of drug administration cycles comprising a duration of treatment, each cycle comprising administering the individual doses throughout a drug dosing interval comprising one or more days, followed by a respective drug intermission interval comprising one or more days;

the method comprising one or more drug dosing intervals wherein in at least one of the drug dosing intervals comprises administration of a boost dose on a first day and of a maintenance dose on one or more following days of each dosing interval;

wherein each individual dose comprises a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant;

or comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts;

or comprises a liquid or dispersed oral formulation comprising (a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester; or any combination thereof.

In various embodiments, the patient can be chemotherapy-naïve, or, the patient has not previously received platinum-based chemotherapy. In other embodiments, the patient has previously received platinum-based chemotherapy and the cancer is refractory to platinum-based chemotherapy reagents, or the patient has previously received platinum-based chemotherapy and the cancer responded but has recurred within 6 months following cessation of the chemotherapy. By platinum-based chemotherapy is meant chemotherapy using an organoplatinum anticancer agent other than picoplatin.

In various embodiments, the method of the invention comprises a first-line treatment. In other embodiments, the method of the invention comprises a second-line or third-line treatment.

In various embodiments, the invention can further comprise treatment with non-platinum based chemotherapy, examples of which are shown in Table 1. For example, in various embodiments, the cancer is prostate cancer and the non-platinum based chemotherapy comprises taxotere; or the cancer is colorectal cancer and the non-platinum based chemotherapy comprises irinotecan; or the cancer is breast cancer and the non-platinum based chemotherapy comprises taxol; or the cancer is ovarian cancer and the non-platinum based chemotherapy comprises liposomal pegylated doxorubicin (Doxil®).

In various embodiments, the invention can further comprise treatment with ionizing radiation, such as X-ray, gamma rays, proton beam, meson beam, or radioisotope radiation from external or implanted source.

The invention in various embodiments further provides a process for preparing an encapsulated unit dosage form for picoplatin, the unit dosage form being adapted for oral administration of the picoplatin, the process comprising preparing a formulation comprising a substantially dry, powder comprising picoplatin, wherein the picoplatin is in physical form a particulate of less than about 10 microns average particle diameter, a substantially water-soluble, water-dispersible, or water-absorbing carbohydrate, and an effective amount up to about 5 wt % of a lubricant; then, disposing the formulation within a substantially water-soluble capsule shell.

The constituent materials used in the process of the invention are as described above for the encapsulated unit dosage form of the invention. The process of the invention comprises preparing the powder of the formulation that is substantially dry, and then filling the capsule shell with the material. The capsule shell is water-soluble and can be light-attenuating, as is described above.

For example, lactose monohydrate, microcrystalline cellulose, and a lubricant such as magnesium stearate can each be ground to pass a 20-mesh screen, then can be blended with the picoplatin particulate together in a granulator. The picoplatin particulate can be prepared earlier by a jet milling process, or can be microcrystalline, or can be lyophilized, or any combination of these processes that provides particulates of the requisite dimensions. A dispersant, for example povidone, such as in the form of a powder that passes a 20-mesh screen, can be added to the mixture in the granulator. Mixing of the solids then can take place using high-shear granulation, so as to form an admixture of the component materials. The picoplatin can be premixed with the carbohydrate, for example lactose, prior to mixing with additional ingredients.

This can serve to reduce the amount of static electricity buildup on the picoplatin particulates. The admixture of the sparingly water-soluble picoplatin (having a solubility of about 1 mg/mL, or about 0.1%, in water) with the water-soluble, water-dispersible or water-absorbing carbohydrate, the lubricant, and optionally with the dispersant, serves to enhance rapid and substantially complete dissolution of an effective amount of the picoplatin in the patient's GI tract.

Following the milling and mixing processes, the formulation can be dried, for example, spread in a thin layer on a tray, which is then held under drying conditions. For example, the powder on the tray can be warmed to a moderate temperature, such as about 40-80° C., and held under a partial vacuum or in the presence of a drying agent, for example, P₂O₅. Residual water can be controlled such that the water content is less than 5 wt %, more preferably less than 1-3 wt %, even more preferably less than 1 wt %, of the solid mixture.

Following drying, additional milling can take place. The bulk material can be sifted through the screen, if desired, to remove any larger particles that may be present. For example, a predominant portion of a sample of a finely particulate material can pass through a 20-mesh screen. Preferably, the bulk of the powder can pass through a 30-mesh screen.

The powder of the formulation can be kept substantially dry through the use of suitable engineering techniques and controls, such as storage under controlled atmosphere, interjection of suitable drying steps into the process for preparation, and storage in the absence of atmospheric moisture. The powder can also be handled under subdued light, in order to minimize the amount of photolytic decomposition of picoplatin, which is well known to be light-unstable. The control of incident light can be carried out by suitable engineering controls, such as processing the material in opaque vessels, conveying it and drying it under cover or in the dark, or the use of safe-lights such as can be used for photographic processing. It is desirable to minimize incident light in carrying out the inventive process.

After the final mixing, drying, and screening steps of the process are carried out, the powder of the formulation is enclosed within the capsule shells, such as by techniques well known in the art. Again, it is preferred that the capsule filling and storage operations be carried out under subdued light and substantially dry conditions.

The invention provides one or more of dosage forms packaged with instruction materials regarding administration of the dosage form, or with instruction materials that comprise labeling means, e.g., labels, tags, CDs, DVDs, cassette tapes and the like, describing a use of the dosage form that has been approved by a government regulatory agency.

The dosage form can include means that provides identifying information useful to a care provider, such as a physician or a nurse, that can include the identity, concentration, expiration date. This can serve to avoid medical mistakes and to provide an additional level of assurance to the care provider and to the patient that the correct medication is being administered. The identifying information can be in a non-visual form so that it can be detected in low light, for example, by textural features of the capsule, raised letters signifying picoplatin and the dosage, and the like. Alternatively, the capsule can be colored in a manner that conveys dosing information or to identify the contents. For example, if a treatment session will use three capsules, the capsules can be coded, such as with different colors, to indicate to the care provider the relative position of a given capsule in the treatment sequence, first, second or third. This serves to avoid medical mistakes such as over- or under-dosing as could occur if the care provider loses count of the capsules administered to a patient in a treatment session.

As a light-sensitive compound, picoplatin and its solutions are protected from light exposure, for example, by packaging in opaque materials. Thus, dosage forms of the present invention can be shielded from light by secondary packaging that minimizes exposure to visible light.

The unit dosage form of the invention, or the unit dosage form prepared by the method of the invention, can have about a ±10% spread in the actual amount of contained picoplatin relative to the nominal composition. For example, a unit dosage form with a nominal 200 mg weight containing a nominal 50 mg of picoplatin, can have about 45 to 55 mg of picoplatin as measured for that individual sample. The unit dosage form of the invention has low and limited amounts of various impurities; for example it should contain no more than about 1% of each of several possible residual impurities from the manufacture or storage of the picoplatin, such as picoline, potassium tetrachloroplatinate, trichloropicoline platinate or trichloroaminoplatinate.

In various embodiments, the invention provides a process for preparing a tablet unit dosage form of the invention. Tablet oral unit dosage forms include the coated tablets disclosed in Application No. PCT/US2008/001752, filed Feb. 8, 2008, published as WO2008/097661, entitled “Stabilized Picoplatin Oral Dosage Form”, by the inventors herein, which are incorporated herein by reference.

The picoplatin that is contained in the powder of the core of a tablet dosage form of the invention is a particulate of an average particle diameter of less than about 10 microns. The picoplatin particulate can be a micronized material, a microcrystalline material, a lyophilized material, or any combination thereof. The picoplatin can be milled or micronized by jet milling, or by any other process that can provide micronized powders of suitably small average particle diameters. Micronized picoplatin, due to the favorable surface area to mass ratio that results from the presence of fine particles, aids in the rapid and complete dissolution of an effective amount of the picoplatin in the patient's GI tract after administration of the dosage form. Micronized picoplatin can be composed of crystalline or amorphous solid picoplatin.

The picoplatin particulate can also be a microcrystalline solid, wherein the powder is composed of crystals of appropriately small physical dimension. Microcrystalline materials can be formed, as is known in the art, by precipitation of a solid from a solution by addition of a liquid in which the material is insoluble, for example with high shear or agitation.

The picoplatin particulate can also be a lyophilized powder, such as is formed by lyophilization of a solution of the picoplatin. The picoplatin particulate can also have been formed by any combination of the above-listed methods of forming fine particulates; for example, a microcrystalline material can be micronized such as by jet milling to reduce particle size, or a material that has been recovered from an aqueous solution by lyophilization can be micronized, and so forth.

The mixture of the picoplatin, the carbohydrate, the lubricant, and any other ingredients that may be present is also in the form of a powder, but is not as fine a powder as the picoplatin particulate. The powder can be a mixture of picoplatin particulates and particles of the other ingredients, or, preferably, the particles making up the powder can have incorporated within substantially every one of them a plurality of the fine textured picoplatin particulates dispersed within particles of the other components such as the carbohydrate. It is preferred that the mixture be an intimate mixture, where picoplatin particulates are closely mixed with the additional ingredients of the formulation, as the greater is the surface area of the component picoplatin particles, and the more intimately these picoplatin particles are mixed with the carbohydrate and with the optional dispersant or disintegrant, the more rapidly and completely the picoplatin will dissolve or disperse after administration of the tablet to the patient. Rapid and complete dissolution of the picoplatin is generally desirable in terms of providing a maximally effective treatment to the patient.

The powder of the core is in a substantially dry form; the water content of ingredients such as carbohydrates and dispersants is controlled to minimize the wt % of water in the formulation. Water, under some conditions, can react with picoplatin, resulting in decomposition. Therefore, the water content of the dosage form is preferably limited to less than 5 wt %, preferably less than 1-3 wt %, and more preferably to less than 1 wt % of the composition. It is understood that certain carbohydrates, for example lactose, may exist in the form of a hydrate, such as a monohydrate which contains 5 wt % water; such hydrates may be used, but exogenous water is preferably excluded as much as is practicable.

The picoplatin, which makes up at least about 10 wt % of the core and can make up to about 60 wt % of the core, is preferably anhydrous, and is handled under conditions during the formulation processes to maintain its dry state. Dryness can be maintained through use of suitable engineering controls, such as operation under a dry, inert atmosphere, as is well known in the art.

The filler, which comprises about 40-80 wt % of the core, comprises a carbohydrate. Suitable carbohydrates can be selected from a group consisting of a monosaccharide, a disaccharide, a sugar alcohol, a cellulose, a modified cellulose and mixtures thereof. Carbohydrates are water-soluble, water-dispersible, or are water-absorbing, that is, the fillers either dissolve completely in water, freely disperse in water, or are sufficiently hydrophilic to absorb substantial amounts of water within their structure. For example, fructose is water-soluble, certain hemicelluloses are water-dispersible, and cellulose is water-absorbing. More than one carbohydrate can be present in the dosage form. The total carbohydrate is preferably present at about 40-80 wt % of the formulation.

The formulation of the invention includes a lubricant in an effective amount. A lubricant, for example the salt of a fatty acid, more specifically magnesium stearate, can serve as a processing aid in handling the powder of the core, in particular the sub-10 micron picoplatin powder, by assisting in avoidance of particle clumping, such as during milling operations. A lubricant can be present at up to about 5 wt % of the core.

A dispersant, which serves to enhance the dispersal of the tablet core in an aqueous medium, such as in the GI tract of a patient, facilitates rapid dissolution. The dispersant tends to assist in dispersion of the particles when they first encounter the aqueous medium, thus helping to preserve the favorable surface area to mass ratio of the fine picoplatin powder. An example of a dispersant is cross-linked sodium carboxymethyl cellulose, also known as croscarmellose. Another example is povidone, also known as polyvidone, poly(vinylpyrrolidone), or PVP. The formulation can comprise about 5-10 wt % of the dispersant. More than one dispersant can be present in the dosage form.

The dosage form can include other ingredients, but does not include redox-active metal salts, and preferably does not include oxidants, or compounds comprising halo, ═NH, —NH₂, or —SH moieties. Other components preferably lacking such groups can be included. For example, anti-oxidants can be included. Examples include BHA or BHT. Colorants, such as food dyes, can be included.

Thus the ratio of picoplatin to carbohydrate filler to dispersant (if present) to lubricant is about 1:1.5-3.0:0.1-0.3:0.25-0.1. In one embodiment, the dosage form comprises about 200 mg of the core material, comprising about 50 mg of micronized picoplatin, about 116 mg of lactose, about 20 mg of microcrystalline cellulose, about 8 mg of croscarmellose sodium, about 4 mg of povidone, and about 2 mg of magnesium stearate, as a compacted admixture, covered by the coating. The coating can be opaquified, as by incorporation of CaSO₄ into the coating.

The invention in various embodiments further provides a process for preparing an oral dosage form adapted for use in a method of the present application comprising a liquid or dispersed formulation comprising a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester, or any combination thereof. See, for example, Application No. PCT/US2008/008669, filed Jul. 16, 2008, published as WO 2009/011861, entitled “Oral Formulations for Picoplatin”.

An embodiment of the self-emulsifying picoplatin formulation can include an oil, and an emulsifier including a lecithin, a surfactant, a PEG, or any combination thereof. Preferably, the self-emulsifying formulation includes at least about 10% w/w of the picoplatin, although it can include lesser amounts of picoplatin, for example, 5% w/w of the picoplatin. The inventive self-emulsifying formulation can also include a first solvent in which picoplatin is at least sparingly soluble, provided that the first solvent is not DMSO. As disclosed hereinbelow, picoplatin is unstable in DMSO, perhaps due to oxidation of the picoplatin by the DMSO. The first solvent can be a dipolar aprotic solvent, a polyethylene glycol, or a polyethyleneglycol ether, a polyethyleneglycol derivative of a mono- or a di-glyceride, or any combination thereof. The dipolar aprotic solvent can be NMP. Preferably the dipolar aprotic solvent, particularly if it is NMP, is substantially free of amine contaminants.

For example, the first solvent can be a polyethyleneglycol derivative of a mono- or a di-glyceride, such as Gelucire 40/14® or Gelucire 50/13®. The picoplatin can be dissolved in the Gelucire held above Gelucire's melting point, i.e., 40° C. for Gelucire 40/14, or 50° C. for Gelucire 50/13. The solution of the picoplatin in the melted Gelucire can then be mixed with other components in the second solvent to form a substantially homogenous second solution. The Gelucire (polyethyleneglycol derivative of a mono-glyceride, i.e., a PEG-ylated monoglyceride) is itself a surfactant; thus mixing the Gelucire solution of the picoplatin with the oil in the second solvent, followed by removal of the second solvent, can provide the self-emulsifying formulation of the invention, wherein the Gelucire serves both as the first solvent and as the emulsifier. Alternatively, lecithin, PEG, another surfactant, or any combination thereof, can also be mixed with the second solvent to provide a substantially homogeneous solution, from which the second solvent is removed to provide the present self-emulsifying formulation.

The self-emulsifying formulation includes an oil, wherein the oil is a medium chain triglyceride, castor oil, a medium chain mono-glyceride, a medium chain di-glyceride, an edible vegetable oil such as peanut oil, cottonseed oil, or soybean oil, or any combination thereof. Alternatively, the oil can be other than a glyceride; for example, the oil can be a hydrocarbon oil or a silicone oil.

The self-emulsifying formulation includes an emulsifier. For example, the emulsifier can contain a lecithin. The lecithin can be a high phosphatidylcholine content lecithin, a low phosphatidylcholine content lecithin, or any combination thereof.

The emulsifier can also include a surfactant, such as Labrasol® (a mixture of glycerides and PEG-ylated materials), Cremophor RH40® (a PEG-ylated glyceride), Cremophor ELP® (a PEG-ylated glyceride), Gelucire 44/14® (a PEG-ylated glyceride), Polysorbate 80 HP® (a PEG-ylated fatty ester of sorbitan), or Vitamin E TPGS (a PEG-ylated tocopherol succinate), or any combination thereof. Gelucire can be both the first solvent and the emulsifier of the inventive self-emulsifying formulation.

The present self-emulsifying formulation can contain a PEG, such as PEG-400. PEG compounds are typically water-soluble, but also can stabilize hydrophobic materials in aqueous media.

For example, the formulation can be prepared by dissolving picoplatin in a first solvent other than DMSO to provide a picoplatin solution, then adding an oil, and an emulsifier comprising a lecithin, a PEG, or a surfactant, or any combination thereof; then, adding a second solvent to dissolve the picoplatin solution, the oil, and the emulsifier, providing a substantially homogeneous second solution; then, evaporating at least the second solvent and, optionally, the first solvent, from the homogeneous solution to provide the self-emulsifying formulation.

The first solvent can be a dipolar aprotic solvent, a polyethylene glycol, or a polyethyleneglycol ether, a polyethyleneglycol derivative of a mono- or di-glyceride, or any combination thereof. The dipolar aprotic solvent can be NMP. Preferably the dipolar aprotic solvent, particularly if NMP, is substantially free of amine contaminants. DMSO is not suitable as the first solvent, due to the instability of picoplatin in DMSO. A solution of a preselected amount of picoplatin for the batch formulation being prepared is dissolved in the first solvent, then the emulsifier is added. The emulsifier can include a lecithin, a PEG, a surfactant, or any combination thereof. The oil can be a medium chain triglyceride, castor oil, a medium chain mono-glyceride, a medium chain di-glyceride, or any combination thereof. The lecithin can be a high phosphatidylcholine content lecithin, a low phosphatidylcholine content lecithin, or any combination thereof. The PEG can be PEG-400. The surfactant can be Labrasol, Cremophor RH40, Cremophor ELP, Gelucire 44/14, Polysorbate 80 HP, or Vitamin E TPGS, or any combination thereof.

Then, a second solvent is added to provide a substantially homogenous second solution, at or near room temperature, although some heating can be used to assist dissolution of all components. Then, the second solvent is removed from the homogenous solution. A suitable second solvent is ethanol, which can be removed under reduced pressure at or near room temperature, although elevated temperatures can also be used. The evaporation can continue such that the first solvent is also removed, although the first solvent or portions of it can remain in the formulation. The residue is a self-emulsifying formulation of the invention, which can be liquid, solid or semi-solid. This material can be filled into hard or soft gelatin capsules for administration to a patient. The self-emulsifying formulation is adapted to aid in dissolution of the picoplatin in the gastrointestinal (GI) tract of the patient, and thus provide for enhanced uptake into the bloodstream compared to the same dose of picoplatin administered as a pure solid.

In another embodiment of the invention, a stabilized nanoparticle preparation of picoplatin is provided that possesses a greatly increased surface area and thus an improved dissolution rate relative to solid crystalline picoplatin. The picoplatin nanoparticles are stabilized with organic materials. For example, the picoplatin nanoparticles can be stabilized with casein, a caseinate, or lecithin, or any combination thereof. Casein and caseinates are proteins found in milk that serve to stabilize butterfat droplets in the aqueous medium. In the present stabilized nanoparticle formulation, the casein or caseinates, or both, can stabilize the sub-micron size picoplatin particles and inhibit re-aggregation of the particles. Also, lipid compositions such as lecithin can be used to stabilize the picoplatin nanoparticles. Preferably, the formulation contains at least about 10% w/w of the picoplatin on a dry weight basis, although the formulation can include a lesser amount of picoplatin, for example, at least about 5% w/w of picoplatin, on a dry weight basis, or an intermediate weight. The formulation can provide improved oral availability of the picoplatin relative to an equivalent dose of solid picoplatin such as in a tablet, or to an equivalent dose of picoplatin in a simple solution such as in water or normal saline solution, that is orally ingested.

The picoplatin nanoparticles can be prepared by a process comprising high-shear mixing or microfluidization. Solid picoplatin, for example picoplatin in crystalline form, can be mixed in an aqueous medium with a stabilizer such as casein, using microfluidization conditions or high-shear conditions, until the average particle diameter of the solid picoplatin is less than about one micron as determined by laser light scattering spectroscopy, or, alternatively, until crystalline picoplatin is observed to be largely absent using an optical microscope with a polarized light filter lens. The average particle diameter can be even smaller; for example the picoplatin nanoparticles can have an average particle diameter of less than about 0.5 micron; of less than about 0.25 micron; or of less than about 0.15 micron.

An embodiment of the invention also provides a method of preparation of the stabilized picoplatin nanoparticles. The method includes mixing a stabilizer and an aqueous medium under high-shear conditions or microfluidization conditions to obtain a uniform dispersion, then adding solid picoplatin, and then continuing mixing under these conditions until an average particle size of the picoplatin is less than about one micron or until crystalline particles are substantially absent, or both, to provide a suspension of the stabilized picoplatin nanoparticles. The stabilizer can be casein, a caseinate, or a lecithin. The average picoplatin particle diameter can be less than about 1 micron, or less than about 0.5 micron, or less than about 0.25 micron, or less than about 0.15 micron.

The suspension of stabilized picoplatin nanoparticles can then be dried to provide a solid material, for example by freeze-drying, to provide a substantially dry solid. By this method, a solid formulation that can be filled into gelatin capsules for oral administration to a patient can be obtained. The picoplatin content of the substantially dry solid can be at least about 10% w/w, or at least about 5% w/w.

In another embodiment of the invention, a dispersion of solid picoplatin in a solid water-dispersible material (matrix) is provided. The inventive solid dispersion can be prepared by a process comprising dispersing of the picoplatin in a melt of the water-dispersible matrix material that then is cooled and solidified. Preferably, the formulation contains at least about 10% w/w of the picoplatin, although the formulation can include a lesser amount of picoplatin, for example, at least about 5% w/w of picoplatin. The water-dispersible matrix material can include Gelucire 50/13, Gelucire 44/14, Poloxamer 188, SPAN 60, PEG-8000, Kollidon K-90, Vitamin E TPGS, or Compritol 888, or any combination thereof, definitions of which are provided herein. The Gelucire and Compritol materials are PEG-ylated glycerides of fatty acids. Poloxamer is a polyethyleneglycol-polypropyleneglycol copolymer. Span is a monostearate ester of sorbitan, and Kollidon is a poly-vinylpyrrolidone. Vitamin E TPGS is a PEG-ylated toxopherol succinate.

The water-dispersible matrix material is at least dispersible in water, not phase-separating spontaneously, and can be completely water-soluble. The matrix material is preferably a solid at about 20° C. to about 37° C. The melt of the water-dispersible matrix material can be held at a temperature of about 40° C. to about 160° C. during dispersion of the solid picoplatin. The step of dispersing the picoplatin in the melt can involve dissolving the picoplatin in the melt to provide a homogenous melt. The homogeneous melt can include Gelucire 50/13, Gelucire 44/14, Compritol 888, or Vitamin E TPGS. The melt is then cooled and solidified to provide the inventive solid dispersion. The formulation can provide improved oral availability of the picoplatin relative to an equivalent dose of solid picoplatin such as in a tablet, or to an equivalent dose of picoplatin in a simple solution such as in water or normal saline solution, that is orally ingested.

In an embodiment of the invention, a nanoparticulate picoplatin suspension in a medium chain triglyceride (MCT oil) or in a fatty ester is provided. The nanoparticulate picoplatin comprises picoplatin particles of less than 1 micron average particle diameter, suspended in the MCT oil or fatty ester. The nanoparticulate picoplatin can make up about 20% up to about 70% by weight of the composition. The MCT oil can be a triglyceride ester of a medium chain fatty acid, or of a combination of different medium chain fatty acids. For example, the MCT oil can be tricaprylglyceride (trioctanoylglyeride) or can be a mixed caprylic/capric (octanoyl/decanoyl) glyceride. All three glycerin hydroxyl groups are acylated in the MCT oil. An example of an MCT oil is a Miglyol brand (Sasol) MCT oil, such as Miglyol 812. Alternatively, the nanoparticulate picoplatin suspension can include a fatty ester. An example is ethyl oleate. The suspension can further contain a lecithin, i.e., a phospholipid. An example is the brand Phospholipon 90G (American Lecithin). The suspension can further contain a sugar ester surfactant, such as a sorbitan ester. An example is sorbitan mono-9-octadecanoate PEG ether (sold under the brand name Sorbate 80).

It is well known that picoplatin can be active against tumors that possess, or have developed, resistance to “first-generation” or “second generation” organoplatinum anti-cancer drugs such as cisplatin and carboplatin. For example, the oral dosage form of the invention or prepared by the process of the invention can be used to treat patients with hematological and non-hematological malignancies, particularly non-hematological malignancies, such as patients with solid malignant tumors, in particular, those patients whose solid tumors are cisplatin, oxaliplatin, or carboplatin refractory. Specific types of solid malignancies that can be treated with the oral dosage form of picoplatin of the invention, or with a picoplatin oral dosage form prepared by the process of the invention, include without limitation, lung cancer, including small cell lung cancer, non small cell lung cancer, head and neck cancer, GI/stomach cancer, skin cancer, ovarian cancer, kidney cancer, bladder cancer, mesothelioma, prostate cancer, including hormone-refractory prostate cancer, cervical/uterine cancer, liver cancer, testicular cancer, pancreatic cancer, colorectal cancer, sarcomas, breast cancer, carcinoid tumors, bone-associated cancers, leukemias, lymphomas (NHL) and the like.

In various embodiments, the method of treatment of the invention can further include orally or parenterally administering, preferably sequentially (before or after) or concurrently (including simultaneously or overlapping), at least one additional medicament and/or anti-cancer therapy, such as radiation therapy, with a unit dosage form or a plurality of unit dosage forms comprising picoplatin, such as the unit dosage form(s) of the invention or prepared by the method of the invention. The additional medicament can be an anti-cancer medicament, preferably a non-Pt containing medicament, and may be administered orally or intravenously.

For example, an additional anti-cancer medicament can comprise, without limitation, a taxane (e.g., paclitaxel or docetaxel), a growth factor receptor inhibitor (e.g., an antibody such as bevacizumab or cetuximab or AZD2171 (Recentin)), an anti-metabolite (capecitabine, gemcitabine or 5-FU with or without leucovorin), a PK inhibitor (e.g., sorafenib tosylate), an anthracyclin (amrubicin, doxorubicin or liposomal doxorubicin), a vinca alkaloid or an alkylating agent, including melphalan and cyclophosphamide. Useful agents include the platinum and non-platinum anticancer drugs disclosed in U.S. patent application Ser. Nos. 10/276,503, filed Sep. 4, 2003; 11/982,841, filed Nov. 5, 2007; 11/935,979, filed Nov. 6, 2007; 11/982,839, filed Nov. 5, 2007; in U.S. Pat. Nos. 7,060,808 and 4,673,668; in PCT WO/98/45331 and WO/96/40210, each of which are incorporated by reference herein. Alternatively, the additional medicament is a non-platinum containing anti-cancer agent, can be selected to treat a complication of the cancer, or to provide relief to a subject from at least one symptom of the cancer.

Preferred anti-cancer medicaments are those that can be administered orally, in effective doses, such as those listed on Table 1, below.

TABLE 1
Orally Administrable Agents

Altretamine
Anagrelide
Anastrozole
(ZD1033)
Bexarotene
Bicalutamide
Capecitabine
Clodronic acid
Cytarabine ocfosfate
Dasatinib
Dutasteride
Erlotinib
Exemestane
Fadrozole
Finasteride
Fludarabine
Gefitinib
GMDP
HMPL 002
Hydroxycarbamide
Ibandronic acid
Idarubicin
Imatinib
Lapatinib
Lenalidomide
Letrozole
Osaterone
Polysaccharide K
Prednimustine
S1
(Gimeracil/oteracil/tegafur)
Sobuzoxane
Sorafenib
Sunitinib
Tamibarotene
Tamoxifen
Tegafur/uracil
Temozolomide
Thalidomide
Topotecan
Toremifene
Treosulfan
Trilostane
Ubenimex
Vinorelbine
Vorinostat

Preferred agents are oral formulations of Altretamine (alkylating agent) (Hexalen®), Capecitabine (anti-metabolite) (Xeoda®), Dasafinib (TK inhibitor) (Spryce®), Erlotinib (EGF receptor antagonist) (Tarceva®), Gefitinib (EGF inhibitor) (Iressa®), Imatinib (TK inhibitor) (Gleevec®), Lapatinib (Tycerb®) (EGFR inhibitor, Her2 inhibitor), Lenalidomide (TNF antagonist) (Revlimid®), Sunitinib (TK inhibitor) (Sutent®), S-1 (anti-metabolite), Sorafenib (angiogenesis inhibitor), Tegafur/Uracil (anti-metabolite) (UFT), Temozolomide (alkylating agent) (Temodar®), Thalidomide (Thalomid) (angiogenesis inhibitor), Topotecan (Hycamptin®), Vinorelbine (Navelbine®), and Vorinostat (HDI) (Zolinza®).

In various embodiments, a method of the invention can comprise administration of a non-platinum anti-cancer agent comprising an alkylating agent, an alkyl sulfonate, a nitrogen mustard, a nitrosourea, an antineoplastic alkaloid, a taxane, an antineoplastic antibiotic, an intercalating agent, an antineoplastic antimetabolite, a folate antagonist, an aromatase inhibitor, a kinase inhibitor, a growth factor inhibitor, an antiinflammatory agent, an anti-topoisomerase, an anthracycline, an anti-angiogenic agent, an immunotherapy adjuvant, a protein kinase C inhibitor, or an ether lipid.

In various embodiments, a method of the invention can comprise administration of a non-platinum anti-cancer agent comprising paclitaxel, vinorelbine, gemcitabine, irinotecan, docetaxel, doxorubicin, dacarbazine, dacarbazine, procarbazine, cyclophosphamide, ifosfamide, methotrexate, mercaptopurine, 6-thioguanine, capecitabine, fluorouracil, cytosine arabinoside, brequinar, tamoxifen, imatinib, alpha-interferon, triphenylthio-phosphoramide, altretamine, topotecan, daunomycin, vinblastine, vincristine, methotrexate, paclitaxel, fluoroorotic acid, N-(phosphonyl)L-aspartic acid, tomudex, camptothecin, octadecylphosphocholine, teniposide, mitomycin, indoloquinones, ormaplatin, staurosporine, bryostatin, mitoxantrone, oligonucleotides, polynucleotides, DNA, 5-azacytidine, cladribine, fludarabine, fluorodeoxyuridine, 6-mercaptopurine, 5-deoxyfluorouridine, ftorafur, 5-deoxy-5-fluorocytidine, 5-aza-cystine arabinoside, troxacitabine, or pentostatin.

The invention in various embodiments further provides a kit comprising packaging containing separately packaged, a sufficient number of the unit dosage forms of the capsules or tablets of the invention or capsules or tablets prepared according to the method of the invention to provide for a course of treatment. A kit can further include instructional materials, such as instructions directing the dose or frequency of administration. For example, a kit can comprise sufficient daily doses for a prolonged period, such as a week or a plurality of weeks, or can comprise multiple unit dosage forms for a single administration when the dose is to be repeated less frequently, such as a daily dose. The multiple unit dosage forms can be packaged separately, but in proximity, as in a blister pack. The kit can also include separately packaged, a plurality of unit dosage forms of the non-platinum containing anti-cancer agent, preferably oral unit dosage forms, including capsules, tablets, or liquid or dispersed formulations.

In various embodiment, the invention provides a use of picoplatin in the manufacture of a medicament adapted for the treatment of cancer, wherein a bioavailability of the picoplatin to a patient after oral ingestion of the medicament is greater than about 50%, wherein the medicament comprises a unit dosage form comprising an effective amount of less than or equal to 200 mg of picoplatin, or less than or equal to 100 mg of picoplatin, or less than or equal to 50 mg of picoplatin, the dosage form being adapted for oral administration of the picoplatin, comprising a substantially water-soluble capsule shell, the dosage form being any of the embodiments of an oral dosage form of the invention as described above.

### EXAMPLES

### Example 1

### Oral Bioavailability Study

The population enrolled were subjects with advanced non-hematological malignancies for whom no standard therapy exists and for whom treatments with single agent picoplatin is appropriate. Subjects may have previously received a platinum agent and be considered “platinum refractory” (e.g., subjects with lung cancer, head and neck cancer, ovarian cancer or other malignancies often treated with platinum-based chemotherapy) or may not have received prior platinum-based chemotherapy (e.g., subjects with sarcomas, breast cancer, carcinoid tumors, etc).

The Study design is a randomized, two-period crossover, open label study in which a single dose (Cycle 1) of picoplatin is given either IV or PO, followed 4 weeks later by a single dose (Cycle 2) of picoplatin given by the route not used for Cycle 1.

The IV dose was 120 mg/m² administered over one hour. This dose is extrapolated from the maximum tolerated dose in heavily pre-treated subjects, likely to be characteristic of the patient population to be studied.

Oral dose levels studied sequentially (6 subjects per dose level) in the absence of dose limiting toxicity: 50 mg, 100 mg, 200 mg, 300 mg, or 400 mg total dose per subject. Assuming an average BSA of 1.7 m², these doses are equal to approximately mean doses of 29, 59, 118, 176, and 235 mg/m².

Blood and urine samples, taken at specified time points after each dose of study drug, are analyzed for total platinum concentrations in plasma (bound platinum) and plasma ultrafiltrate (unbound platinum).

Pharmacokinetic parameters observed are shown below in Table 3. Measurements of platinum concentrations in plasma and in plasma ultrafiltrate (PUF) for the dosage range of 50-400 mg for oral administration, and of 120 mg/m² for IV administration, are shown. The IV concentrations observed in plasma and plasma ultrafiltrate were used as defined value of 100% against which the plasma and PUF platinum values for the orally administered picoplatin were measured.

Capsules, which are opaque white, are sealed with a green band. The appropriate number of capsules for the prescribed dose are removed from the bottle and placed in a drug envelope (or other vessel) such that the subject can easily slide the capsules into the mouth without touching the capsules. Protection from light while transporting capsules to the subjects for ingestion is preferred. The composition of a capsule containing 50 mg of picoplatin is given in Table 2, below.

TABLE 2
Composition of Picoplatin Gelatin Capsule 50 mg

| Ingredient | Amount (mg) | Function |
|---|---|---|
| Picoplatin | 50 | Active Ingredient |
| Lactose 450# | 116 | Filler |
| Avicel PH101 | 20 | Binder/Filler |
| Croscarmellose Sodium | 8 | Disintegrant |
| Polyvidone | 4 | Disintegrant/binder |
| Magnesium Sterate | 2 | Glidant |

Picoplatin capsules are taken orally with the subject swallowing the entire prescribed dose over 5 minutes with 240 mL (8 ounces) of water (after consumption of clear liquids and anti-emetic therapy only during the preceding 4 hours).

Premedication anti-emetics include dexamethasone, 8-12 mg, (or equivalent corticosteroid) and a 5-HT₃ receptor antagonist given PO or IV immediately prior to the study drug. Subjects also receive anti-emetic therapy as needed for several days following treatment, e.g., oral lorazepam, prochlorperazine, or equivalent as clinically indicated. Each subject must receive the same anti-emetic regimen (drugs, dose and route) during Cycles 1 and 2.

### Results

Results of the bioavailability study are shown in Table 3. Analytical results show pharmacokinetic data as obtained from plasma and from plasma ultrafiltrate (PUF), including Cmax, Tmax, AUC, and F(%) (total bioavailability). Picoplatin was administered in the doses and in the forms noted, the cohort size for each study group being indicated in the first column.

The control subjects received an intravenous dose of 120 mg/m² picoplatin, were used to define a 100% bioavailability level of platinum in plasma and PUF.

The test subjects received oral doses of picoplatin using the dosage form described herein, with the total oral dose of 50, 100, 200, 300, or 400 mg picoplatin being indicated for each cohort. At the 50 mg/patient dose level the total bioavailability F is shown to be 79±24% when measured in plasma, and 110±23%when measured in PUF; i.e., near quantitative bioavailability in the blood stream, within experimental error. At the 100 mg/patient dose level, total bioavailability is 86±18% in plasma, and 116±28% in PUF, again, near quantitative bioavailability, within experimental error. Oral bioavailability at these dose levels provides for more efficient use of picoplatin in treatment of cancer patients.

These data unexpectedly are in sharp contrast to the picoplatin total bioavailability seen at oral dose levels of 200, 300, and 400 mg. The highest total bioavailability seen either in plasma or in PUF in those cohorts amounts to only 44±4% at the 200 mg level in PUF; in plasma at this dose level the total bioavailability is only 39±15% in plasma. At the higher dose levels, the bioavailability in all cases is under 30%.

It was not anticipated, and is surprising, that such a sharp distinction was observed in the bioavailability with increasing dose. This observation by the inventors herein now provides a basis for efficient use of picoplatin in treatment of cancer using the present method and oral route of administration.

It should be noted that all doses were well tolerated and no serious adverse events were noted following the oral dose. There was no evidence of myelosupproession after oral dosing.

Graph 1, below, shows a simulation of blood levels in patients using a method of the invention, whereby a series of successive doses of picoplatin at doses of 100 mg/day, using the dosage form of the invention, over a period of 5 days per week for two weeks, followed by a drug intermission interval wherein no picoplatin was administered, lasting about 9 days, followed by two additional weeks of 5 day/week dosing with 100 mg/day of the dosage form. Table 4, below, provides numerical projected pharmacokinetic data for the oral dosing using this regimen.

TABLE 3
Pharmacokinetic Parameters in Plasma and Plasma Ultrafiltrate (PUF)

| | | | | | | Terminal | AUC_{inf} | |
|---|---|---|---|---|---|---|---|---|
| | Cohort/ | | Cₘₐₓ | Tₘₐₓ | Cl_{obs/}Cl_F | Half-life | (hr * μg/L) × | |
| | Dose (n) | Route | (μg/L) | (hr) | (L/hr) | (hr) | 1000 | F (%) |
| PLASMA | 50 mg/ | Oral | 199 ± 71 | 3 ± 1 | 1.3 ± 0.5 | 163 ± 32* | 22.1 ± 8.5 | 79 ± 24 |
| | patient | | | | | | | |
| | (5) | | | | | | | |
| | 100 mg/ | Oral | 351 ± 132 | 4 ± 2 | 1.2 ± 0.3 | 181 ± 34* | 46.9 ± 11.7 | 86 ± 18 |
| | patient | | | | | | | |
| | (6) | | | | | | | |
| | 120 mg/m² | IV | 5477 ± 1465 | 1 ± 0 | 0.5 ± 0.2 | 122 ± 9 | 229.5 ± 44.9 | 100 |
| | (6) | | | | | | | |
| | 200 mg/ | Oral | 803 ± 303 | 4 ± 2 | 1.7 ± 1.5 | 130 ± 30 | 99.4 ± 54.0 | 39 ± 15 |
| | patient | | | | | | | |
| | (5) | | | | | | | |
| | 120 mg/m² | IV | 5082 ± 1186 | 1 ± 0 | 0.5 ± 0.2 | 125 ± 10 | 288.4 ± 36.7 | 100 |
| | (6) | | | | | | | |
| | 300 mg/ | Oral | 745 ± 182 | 5 ± 2 | 1.8 ± 0.3 | 134 ± 20 | 93.8 ± 23.3 | 28 ± 16 |
| | patient | | | | | | | |
| | (5) | | | | | | | |
| | 120 mg/m² | IV | 4390 ± 1061 | 1 ± 0 | 0.6 ± 0.2 | 116 ± 16 | 214.8 ± 31.0 | 100 |
| | (6) | | | | | | | |
| | 400 mg/ | Oral | 854 ± 189 | 3 ± 1 | 2.6 ± 1.1 | 108 ± 63 | 90.7 ± 34.4 | 21 ± 6 |
| | patient | | | | | | | |
| | (5) | | | | | | | |
| PLASMA | 50 mg/ | Oral | 132 ± 59 | 2 ± 1 | 17.3 ± 4.6 | 270 ± 92* | 1.6 ± 0.4 | 110 ± 23 |
| ULTRAFILTRATE | patient | | | | | | | |
| (PUF) | (5) | | | | | | | |
| | 100 mg/ | Oral | 227 ± 98 | 3 ± 1 | 16.9 ± 3.9 | 182 ± 628* | 3.3 ± 1.2 | 116 ± 28 |
| | patient | | | | | | | |
| | (6) | | | | | | | |
| | 120 mg/m² | IV | 4245 ± 999 | 1 ± 0 | 12.2 ± 2.6 | 64 ± 12 | 11.0 ± 1.4 | 100 |
| | (6) | | | | | | | |
| | 200 mg/ | Oral | 585 ± 319 | 3 ± 1 | 22.9 ± 5.1 | 77 ± 33 | 4.7 ± 1.1 | 44 ± 4 |
| | patient | | | | | | | |
| | (5) | | | | | | | |
| | 120 mg/m² | IV | 3960 ± 969 | 1 ± 0 | 10.2 ± 4.4 | 67 ± 11 | 15.6 ± 3.4 | 100 |
| | (6) | | | | | | | |
| | 300 mg/ | Oral | 392 ± 180 | 5 ± 2 | 30.8 ± 5.3 | 60 ± 20 | 5.2 ± 0.9 | 27 ± 10 |
| | patient | | | | | | | |
| | (5) | | | | | | | |
| | 120 mg/m² | IV | 3687 ± 1046 | 1 ± 0 | 13.3 ± 4.8 | 65 ± 6 | 11.2 ± 2.5 | 100 |
| | (6) | | | | | | | |
| | 400 mg/ | Oral | 452 ± 196 | 3 ± 1 | 48.6 ± 12.4 | 55 ± 31 | 4.5 ± 1.3 | 21 ± 5 |
| | patient | | | | | | | |
| | (5) | | | | | | | |

TABLE 4
Pharmacokinetic Data from Graph 1

Oral
100 mg × 5 days

| Dosing | AUC | Cₘₐₓ |
|---|---|---|
| Schedule | hr * μg/L | μg/L |
| 1^{st} Week | 180400 | 994 |
| 2^{nd} Week | 250700 | 1380 |

| | |
|---|---|
| 3^{rd} Week | Drug Holiday |

| | | |
|---|---|---|
| 4^{th} Week | 218400 | 1200 |
| 5^{th} Week | 265500 | 1460 |

Graph 1 shows two beneficial effects concerning picoplatin level using a method of the invention: a slower rise to a maximal picoplatin concentration in the blood, occurring stepwise over several days rather than virtually instantaneously as in the case of IV administration, and maintenance of a therapeutic level of picoplatin in the blood over a longer period of time than is achieved with a single IV administration. As shown in Table 4, the AUC and the Cmax values are relatively constant over the five week period.

Graph 2, below, shows another simulation of serum picoplatin levels using the dosing regimens indicated, compared to a single IV administration at the stated level. Table 5 provides a summary of numerical pharmacokinetic data displayed in Graph 2.

TABLE 5
Pharmacokinetic Data from Graph 2

| | IV | | Oral × 5 days | | | | | |
|---|---|---|---|---|---|---|---|---|
| | 150 mg/m² | | 50 mg | | 100 mg | | 200 mg | |
| Dosing | AUC | Cₘₐₓ | AUC | Cₘₐₓ | AUC | Cₘₐₓ | AUC | Cₘₐₓ |
| Schedule | hr * μg/L | μg/L | hr * μg/L | μg/L | hr * μg/L | μg/L | hr * μg/L | μg/L |
| QD | 372987 | 2997 | — | — | — | — | 594600 | 3275 |
| Q8Hr | — | — | 259200 | 1430 | — | — | — | — |
| Q12Hr | — | — | — | — | 349400 | 1930 | — | — |

Graph 2 shows how use of an inventive method of oral dosing, comprising administering a series of oral dosage forms of the invention of the stated amount at the stated frequency can provide a therapeutic level of picoplatin with a lower peak level of picoplatin compared to IV administration, while maintaining therapeutic levels in the bloodstream over a longer period of time. The upper dark stepped line represents the 200 mg 1× daily dose. The middle stepped line represents the 100 mg 2× daily dose and the lower stepped line represents the 50 mg 3× daily dose.

By selection of a dose containing a particular amount of picoplatin of less than about 200 mg, preferably of about 100 mg or less per dose, and selecting a frequency of administration of the dosage form, a total dose per day substantially equivalent to a therapeutic IV dose can be achieved. By combination of the use of the series of successive doses of the oral dosage form and of drug intermission intervals, a relatively steady concentration of picoplatin can be maintained in the patient's bloodstream over a period of weeks or months, while avoiding the high levels or peaks that occur when IV administration is used. This avoidance of high, potentially toxic, levels of picoplatin provides a greater safety margin for the patient in that deleterious side effects are more likely to be avoided, while providing a comparable therapeutic benefit.

This application is related to:

Application No. PCT/US2008/001746, filed Feb. 8, 2008, published as WO2008/097658, entitled “Encapsulated Picoplatin”;

Application No. PCT/US2008/001752, filed Feb. 8, 2008, published as WO2008/097661, entitled “Stabilized Picoplatin Oral Dosage Form”;

Application No. PCT/US2008/008669, filed Jul. 16, 2008, published as WO 2009/011861, entitled “Oral Formulations for Picoplatin”;

Application No. PCT/US2008/008076, filed Jun. 27, 2008, published as WO2009/032034, entitled “Stabilized Picoplatin Dosage Form”;

Application No. PCT/US2009/000770, filed Feb. 6, 2009, published as WO2009/099649, entitled “Use of Picoplatin and Bevacizumab to Treat Colorectal Cancer”;

Application No. PCT/US2009/000773, filed Feb. 6, 2009, published as WO2009/099651 entitled “Use of Picoplatin and Cetuximab to Treat Colorectal Cancer”;

Application No. PCT/US2009/000750, filed Feb. 6, 2009, published as WO2009/099634 entitled “Picoplatin and Amrubicin to Treat Lung Cancer”;

U.S. Ser. No. 10/276,503, filed Sep. 4, 2003, entitled “Combination Chemotherapy”;

U.S. Ser. No. 11/982,839, filed Nov. 5, 2007, entitled “Use of Picoplatin to Treat Small Cell Lung Cancer”;

U.S. Ser. No. 11/982,841, filed Nov. 5, 2007, entitled “Use of Picoplatin to Treat Colorectal Cancer”;

U.S. Ser. No. 11/935,979, filed Nov. 6, 2007, entitled “Use of Picoplatin to Treat Prostate Cancer”;

U.S. Ser. No. 12/367,394, filed Feb. 6, 2009, “Use of Picoplatin to Treat Colorectal Cancer”;

U.S. Ser. No. 12/464,662, filed May 12, 2009, “Use of Picoplatin to Treat Colorectal Cancer”;

U.S. Ser. No. 12/465,563, filed May 13, 2009, “Use of Picoplatin to Treat Colorectal Cancer”;

U.S. Ser. No. 12/635,534, filed Dec. 10, 2009, “Combination Therapy for Ovarian Cancer”;

all of which are incorporated by reference herein in their entireties.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of treating cancer in a human patient afflicted therewith, comprising administering orally to the patient one or more individual doses per day each comprising picoplatin, wherein each individual dose comprises less than about 200 mg of picoplatin, wherein an aggregate daily dose comprises a sum of the one or more individual doses administered within a single day, provided that when more than one individual dose makes up a aggregate daily dose, each individual dose is administered non-concurrently with each other individual dose over the course of the day.

2. The method of claim 1 wherein an oral bioavailability of each individual dose is greater than about 50%.

3. The method of claim 1 wherein a daily average oral bioavailability of the picoplatin to the patient from the daily aggregate dose is greater than about 50%.

4. The method of claim 1 further comprising administration of the individual doses, each comprising a maintenance dose of less than about 200 mg of picoplatin, throughout a drug dosing interval comprising a successive or intermittent plurality of days.

5. The method of claim 1 further comprising quasi-metronomic dosing comprising administering the picoplatin to the patient throughout a plurality of drug administration cycles comprising a duration of treatment, each cycle comprising administering the individual doses, each comprising less than about 200 mg of picoplatin, throughout a drug dosing interval comprising one or more days, followed by a respective drug intermission interval comprising one or more days.

6. The method of claim 4 comprising one or more drug dosing intervals wherein in at least one of the drug dosing intervals further comprises administration of one or more boost doses, each boost dose independently comprising a dose greater than the individual dose of claim 1.

7. The method of claim 6 wherein the boost dose is 200 to 500 mg or incremental quantities therebetween of picoplatin.

8. The method of claim 1 wherein administration of an individual dose comprises administration of one or more dosage forms comprising a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant.

9. The method of claim 1 wherein administration of an individual dose comprises administration of one or more dosages form comprising a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts.

10. The method of claim 1 wherein administration of an individual dose comprises administration of one or more dosages form comprising a liquid or dispersed oral formulation comprising (a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester, or any combination thereof.

11. (canceled)

12. The method of claim 1 wherein each aggregate daily dose is provided by spaced administration of two or three individual doses each comprising less than about 200 mg of picoplatin apiece, or less than about 100 mg of picoplatin apiece.

13. The method of claim 12 wherein the doses are regularly spaced.

14. (canceled)

15. (canceled)

16. (canceled)

17. The method of claim 5 comprising a dosing interval of about 1-30 days followed by a drug intermission interval of about 1-4 weeks.

18. The method of claim 17 comprising a dosing interval of about 1-5 days followed by a drug intermission interval of about 3 weeks.

19. The method of claim 17 comprising a plurality of cycles of the dosing interval followed by the drug intermission interval.

20. The method of claim 17 wherein one or more of the dosing intervals further comprises administration of one or more boost doses of up to 400 mg picoplatin apiece, each boost dose comprising 200-500 mg of picoplatin apiece.

21. The method of claim 2 wherein the oral bioavailability of the picoplatin in the patient after ingestion of each individual dose is greater than about 60-75%, or is greater than about 90%.

22. (canceled)

23. The method of claim 3 wherein the daily average oral bioavailability of the picoplatin to the patient from the daily aggregate dose is greater than about 60-75%, or is greater than about 90%.

24. (canceled)

25. The method of claim 1 wherein the aggregate daily dose of picoplatin is about 10-500 mg, or about 6-294 mg/m².

26. The method of claim 1 wherein the aggregate daily dose of picoplatin is about 25-150 mg, or about 15-88 mg/m².

27. The method of claim 1 wherein each individual dose comprises about 10-180 mg of picoplatin.

28. The method of claim 1 wherein each individual dose comprises 50, 60, 70, 80, 90, 100, 110, 120, 130, 140, 150, 160, 170, 180, or 190 mg, or incremental quantities therebetween, of picoplatin.

29. The method of claim 27 wherein each individual dose comprises about 25-175 mg of picoplatin., or about 50-150 mg of picoplatin, or about 50-100 mg of picoplatin.

30. (canceled)

31. (canceled)

32. The method of claim 4 wherein the successive daily doses comprise daily doses administered for a period of 2 days to 5 months, or the intermittent daily doses comprise doses administered about every other day for a period of 3 days to 5 months

33. The method of claim 5 wherein the drug intermission interval is about 2 days to about 4 weeks, or is about 2 to 3 weeks.

34. (canceled)

35. The method of claim 4 wherein a steady state level of picoplatin at therapeutic levels in the patient's blood is attained in about 3-5 days.

36. The method of claim 1 wherein each individual dose is administered after at least about 4 hours of fasting, or at least about 2 hours of fasting.

37. (canceled)

38. The method of claim 1 wherein one or more individual doses is administered during the night time.

39. The method of claim 5 wherein 2 to about 10 cycles are used, or wherein about 4-6 cycles are used.

40. (canceled)

41. The method of claim 5 wherein the duration of treatment is about 2 weeks to about 40 weeks, or is about 10 weeks to about 30 weeks.

42. (canceled)

43. The method of claim 5 wherein the drug dosing interval and the drug intermission interval are each adjusted in duration based upon an evaluation of severity of picoplatin side-effects in the patient following a first or subsequent administration of picoplatin.

44. The method of claim 5 wherein the aggregate daily dose of picoplatin is adjusted based upon an evaluation of severity of picoplatin side-effects in the patient following a first or subsequent administration of picoplatin.

45. The method of claim 43 wherein the side-effects include neutropenia, thrombocytopenia, anemia, nausea, vomiting, fatigue, neuropathy, diarrhea, leucopenia, or alopecia, or any combination thereof.

46. The method of claim 1 further comprising administering at least one non-platinum anti-cancer agent to the human sequentially or concurrently with the picoplatin.

47. The method of claim 46 wherein the administration is oral.

48. The method of claim 1 further comprising treatment with ionizing radiation.

49. The method of claim 48 wherein the ionizing radiation is X-ray, gamma rays, proton beam, meson beam, or radioisotope radiation from external or implanted source.

50. A unit dosage form adapted for administration of the boost dose of claim 19, comprising 200 mg or more of picoplatin.

51. A unit dosage form of picoplatin for oral administration wherein the dosage form comprises about 0.1 mg to less than about 200 mg of picoplatin, providing an oral bioavailability of at least about 50% to the patient.

52. The unit dosage form of claim 51 comprising a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant.

53. The unit dosage form of claim 51 comprising a solid core comprising about 10 to 60 wt % particulate picoplatin wherein the picoplatin is a particulate of less than about 10 microns average particle diameter, about 40-80 wt% of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts.

54. The unit dosage form of claim 51 comprising a liquid or dispersed oral formulation comprising (a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester, or any combination thereof.

55. The unit dosage form of claim 51 wherein each dosage form comprises about 25-150 mg of picoplatin. or wherein each dosage form comprises about 50-100 mg of picoplatin.

56. (canceled)

57. Use of picoplatin for treatment of cancer, the use comprising administering orally to the patient one or more individual doses per day each comprising picoplatin, wherein each individual dose comprises less than about 200 mg of picoplatin, wherein an aggregate daily dose comprises sum of the one or more individual doses administered within a single day, provided that when more than one individual dose makes up a aggregate daily dose, each individual dose is administered non-concurrently with each other individual dose over the course of the day, wherein an oral bioavailability of each individual dose is greater than about 50%.

58. A method of treating cancer in a human patient afflicted therewith, comprising administering orally to the patient one or more individual doses per day each comprising picoplatin, wherein each individual dose comprises less than about 200 mg of picoplatin, wherein an aggregate daily dose comprises sum of the one or more individual doses administered within a single day, provided that when more than one individual dose makes up a aggregate daily dose, each individual dose is administered non-concurrently with each other individual dose over the course of the day;
wherein an oral bioavailability of each individual dose is greater than about 50%, or wherein a daily average oral bioavailability of the picoplatin to the patient from the daily aggregate dose is greater than about 50%;
the method further comprising administration of the individual doses, each comprising a maintenance dose of less than about 200 mg of picoplatin, throughout a drug dosing interval comprising a successive or intermittent plurality of days;
the method further comprising administering the picoplatin to the patient throughout a plurality of drug administration cycles comprising a duration of treatment, each cycle comprising administering the individual doses throughout a drug dosing interval comprising one or more days, followed by a respective drug intermission interval comprising one or more days;
the method comprising one or more drug dosing intervals wherein in at least one of the drug dosing intervals comprises administration of a boost dose on a first day and of a maintenance dose on one or more following days of each dosing interval.

59. The method of claim 58, wherein each individual dose comprises a substantially water-soluble capsule shell, the capsule shell enclosing a formulation comprising a substantially dry powder comprising about 10 to 60 wt % particulate picoplatin of less than about 10 microns average particle diameter, a substantially water-soluble, water-dispersible, or water-absorbing carbohydrate, and an effective amount of up to about 5 wt % of a lubricant;
or comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts;
or comprises a liquid or dispersed oral formulation comprising (a) a self-emulsifying formulation containing picoplatin, (b) a plurality of stabilized picoplatin nanoparticles, (c) a picoplatin solid dispersion in a water-dispersible matrix material, or (d) a nanoparticulate picoplatin suspension in a medium chain triglyceride or a fatty ester;
or any combination thereof.

60. The method of claim 58 wherein the patient is chemotherapy-naïve.

61. The method of claim 58 wherein the patient has not previously received platinum-based chemotherapy.

62. The method of claim 58 wherein the patient has previously received platinum-based chemotherapy and the cancer is refractory to platinum-based chemotherapy reagents.

63. The method of claim 58 wherein the patient has previously received platinum-based chemotherapy and the cancer responded but has recurred within 6 months following cessation of the chemotherapy.

64. The method of claim 58 comprising first-line treatment.

65. The method of claim 58 comprising second-line or third-line treatment.

66. The method of claim 58 further comprising treatment with non-platinum based chemotherapy.

67. The method of claim 66 wherein the non-platinum based chemotherapy comprises oral administration of an anti-cancer agent.

68. The method of claim 66 wherein the cancer is prostate cancer and the non-platinum based chemotherapy comprises docetaxel; or wherein the cancer is colorectal cancer and the non-platinum based chemotherapy comprises 5-fluorouracil; or wherein the cancer is breast cancer and the non-platinum based chemotherapy comprises taxol, or wherein the cancer is ovarian and the non-platinum based chemotherapy comprises liposomal doxorubicin.

68. The method of claim 58 further comprising treatment with ionizing radiation.

69. The method of claim 68 wherein the ionizing radiation is X-ray, gamma rays, proton beam, meson beam, or radioisotope radiation from external or implanted source.

70. The method of claim 1 or of claim 58 wherein the cancer comprises small cell lung cancer, colorectal cancer, prostate cancer including castration-resistant prostate cancer, or ovarian cancer.
